SEQUENCE LISTING (4 sequences)

SEQ ID NO: 1 - DNA, 26 bp, artificial sequence
  Primer sequence Hu4
atggcgaccctggaaaagctgatgaa

SEQ ID NO: 2 - DNA, 22 bp, artificial sequence
  Hu3-510 Primer sequence
gggcgcctgaggctgaggcagc

SEQ ID NO: 3 - protein, 1497 aa, Rattus norvegicus
MLAKQQMHIDSHEALGVLNTLFEILAPSSLRPVDMLLRSMFITPSTMASVSTVQLWISGILAILRVLISQSTEDIVLSRIQELSFSPYLISCPVINRLRDGDSNPTLGERSRGKQVKNLPEDTFSRFLLQLVGILLEDIVTKQLKVDMSEQQHTFYCQELGTLLMCLIHIFKSGMFRRITAAATRLFTSDGCEGSFYTLDSLNARVRAMVPTHPALVLLWCQILLLINHTDHRWWAEVQQTPKRHSLSCTKSLNPQISAEEDSGSAAQLGMCNREIVRRGALILFCDYVCQNLHDSEHLTWLIVNHIQDLISLSHEPPVQDFISAIHRNSAASGLFIQAIQSRCENLSTPTTLKKTLQCLEGIHLSQSGAVLTLYVDRLLGTPFRALARMVDTLACRRVEMLLAANLQSSMAQLPEEELNRIQEHLQNTGLAQRHQRLYSLLDRFRLSTVQDSLSPLPPVTSHPLDGDGHTSLETVNPDKDWYLQLVRSQCWTRSDSALLEGAELVNRIPAEDMSDFMMSSEFNLSLFAPCLSLGMSEIAGSQKSPLFEAARRVTLDRVTNVVQQLPAVHQVFQPFLPTEPTAYWSKLNDLFGDTTSYQSLTTLARALAQYLVVLSKVPAPLHLPPEKEGHTVKFVVMTLEALSWHLIHEQIPLSLDLQAGLDCCCLALQVPGLWGVLSSPEYVTHTCSLIHCVRFILEAIAVQPGDQLLGPESRSHTPRAVRKEEVDSDIQNLSHITSACEMVADMVESLQSVLALGHKRNSTLPSFLTAVLKNIVVSLARLPLVNSYTRVPPLVWKLGWSPKPGGDFGTVFPEIPVEFLQEKEVLKEFIYRINTLGWTSRTQFEETWATLLGVLVTQPLVMEQEESPPEEDTERTQIHVLAVQAITSLVLSAMAVPVAGNPAVSCLEQQPRNKPLKALDTRFGRKLSMIRGIVEQEIQEMVSQRENTATHHSHQAWDPVPSLLPATTGALISHDKLLLQINSEREPGNMSYKLGQVSIHSVWLGNNITPLREEEWDEEEEEEADAPAPTSPPVSPVNSRKHRAGVDIHSCSQFLLELYSRWILPSSAARRTPVILISEVVRSLLVVSDLFTDVPQFEMMYLTLTELRRVHPSEDEILIQYLVPATCKAAAVLGMDKTVAEPVSRLLESTLRSTHLPSQIGALHGILYVLECDLLDDTVKQLIPVVSDYLLSNLKGIAHCVNIHSQQHVLVMCATAFYLMENYPLDVGPEFSASVIQMCGVMLSGSEESTPSVIYHCALRGLERLLLSEQLSRLDTESLVKLSVDRVNVQSPHRAMAALGLMLTCMYTGKEKASPGRASDPSPATPDSESVIVAMERVSVLFDRIRKGFPCEARVVARILPQFLDDFFPPQDVMNKVIGEFLSNQQPYPQFMATVVYKVFQTLHSAGQSSMVRDWVMLSLSNFTQRTPVAMAMWSLSCLPCQCIYQPMGFCNPSTRHQQDGQTRAGGCEPFLPGCHRLLQTPDRGGIRPQGFPVCV

SEQ ID NO: 4 - DNA, 9929 bp, Rattus norvegicus
ggcacgagcagggttgccatgacggcctcctggaaaaactgatgaaggctttcgagtcgctcaagtcgttccagcagcaacagcagcagcagcagccgccgccgcagccgccaccaccgccgccgccgccgcctcaaccccctcagccgccgcctcaggggcagccgccgccaccaccgccgctgccaggtccggccgaggagccgctgcaccgaccaaagaaggaactctcagccaccaagaaggaccgtgtgaatcactgtctaacaatatgtgaaaacattgtggcacagtctctcagaaattctccagaatttcagaaactcttgggcattgctatggaactgtttctgctgtgcagcgacgatgcgagtagacgtagaatggtggctgatgagtgcctgaacaaagtcatcaaagctttgatggactctaatcttccaaggctacagttagaactctataaggaaattaaaaagaatggtgctcctcgaagtttgcgtgcagctctgtggaggtttgctgagctggctcacctggttcgacctcagaagtgcaggccttatctggtgaatcttcttccatgtttgacccgaacaagcaaacgaccggaggagtcagttcaggagactttggctgcagctgttcctaaaattatggcctcttttggcaatttcgcgaatgacaatgaaattaaggttctattgaaagctttcatagcaaatctgaagtcaagctctcccactgtgcggcggacagcagctgggtcagcagtgagtatctgccagcactctaggaggacacagtacttctacaactggctcctgaatgtgctcctaggtttgctggttcccatggaggaagaccaccccactctcctgatccttggtgtgttgctcacactgaggtgtctagtgcccttgctccagcagcaggtcaaggacacaagtctaaagggcagctttggggtaacacggaaagaaatggaagtctctccttctgcagagcagcttgtccaggtttatgaactgactttgcatcacacacagcaccaagaccataatgtggtgacaggggcattggagctcctgcagcagctcttccgtacccctccacctgagctgctgcaagcactgaccacaccaggagggctcgggcagctcactctggttcgagaggaagccgggggccgaggccgcagcgggagtatcgtggagcttttagctggagggggttcctcatgcagccctgttctctcaagaaagcaaaaaggcaaagtgctcttaggagaggaagaagccttggaggatgactcggagtccaggtcagatgtcagcagctcagcctttgcagcctctgtgaagagtgagattggtggagagctcgctgcttcttcttcgggtgtctccactcccggttctgtaggtcacgacatcatcactgagcagcctcgatcccagcacacacttcaagcagactctgtggatttgtcaggctgtgacttgaccagtgctgctactgatggagatgaggaagacatcttgagccacagctccagccagttcagtgctgttccatccgaccctgccatggacctgaatgatgggacccaggcctcctcacccatcagtgacagttctcagaccaccactgaaggacctgattcagctgtgactccttctgacagttctgaaattgtcttagatggtgctgacagccagtatttaggcgtgcagataggacagccacaggaggaagacagagaagctgcaggtgttctttctggtgaagtctcagacgttttcagaaactcttctctggcccttcagcaggcacacttgttggaaagaatgggtcatagccggcagccttctgacagcagtgttgataagtttgtttcaaaagatgaggttgctgaagctggggacccagaaagcaagccttgccgaatcaaaggtgacataggacagcctaatgatgatgattctgctcctctggtacattgtgtccgtcttttatccgcttcctttttgttaactggcgaaaagaaagcactggttccagacagagatgtgagagtcagtgtgaaggccctggccctcagctgtattggtgcagctgtggcccttcatccagagtcgttcttcagcaaactctacaaagtacctctcagtaccatggaaagtactgaggaacagtatgtctctgacatcctgaactacatcgatcatggagaccctcaggtgcgaggagctactgccattctctgtgggacccttgtctactccatcctcagcaggtcccgtctccgtgttggtgactggctgggcaccatcagggccctgacaggaaatacattttctctggtggactgcattcctttactgcagaaaactttgaaggatgaatcttctgttacttgcaagttggcttgtacagctgtgaggcactgtgtcctgagtctttgcagcagcagctacagtgacttgggattacaactgcttattgacatgctgcctctgaagaacagctcctactggctggtgaggactgaactgctggaaactcttgcagagattgatttcaggctggtgagttttttggaggcaaaagcagaaagtttacaccgaggacctcatcattatacagggtttctaaaactacaagaacgagtactcaataatgtggtcatttatttgcttggagatgaagaccccagggttcgacatgttgctgcgacgacattgacaagacttgtcccaaagctgttttataagtgtgaccaaggacaggctgacccagtcgaggctgtagcaagagatcaaagtagtgtttacctgaagctcctcatgcatgagacccagccaccatcccacttctccgtcagcaccataaccagaatctatagaggctacagcttactaccaagtgtaacagatgtcaccatggaaaacaacctctcaagagtcgttgccgcagtttctcatgaactcattacgtcaactacacgggcactcacatttgggtgctgtgaagccttgtgtgttctttcagccgcctttccagtttgcacttggagtctaggatggcactgtggagtgcccccactgagtgcctctgatgagtccaggaagagctgcactgttgggatggcctccatgattctcaccttgctttcatcagcttggttcccactggatctctcagccattcaggatgccttgattttggctggaaacttgctagcagcgagtgcccccaagtctctgagaagctcatgggcctcggaagaagaaggcagctcagcagccaccagacaggaggagatctggcctgccctgggggatcggactctggtgcccatggtggagcagcttttctcccacctgctgaaggtgatcaatatctgtgctcatgtcttggatgacgagactcctggaccagcaatcaaggcagctttgccttctctcacaaaccccccttctctaagtcctattcgacggaaagggaaggagaaagagcccggagaacaaacatccactccgatgagtcccaagaaaggtggagaggccagtacagcctctcgacagtcagacacctcaggacctgtcacagcgagtaaatcatcttcacttgggagtttctaccatctcccttcctacctcagactgcatgatgtcctgaaagccactcacgccaactataaggtcaccttagatcttcagaacagcactgaaaagtttggggggttcctgcgctctgccttggacgtcctttctcagattctagagctggcgacactgcaggacattggaaagtgtgttgaagaggtccttggatacttgaaatcctgctttagtcgagaaccaatgatggcgactgtctgtgttcagcagctattgaagactctctttgggacaaacttagcctcacagtttgatggcttatcttccaaccccagcaagtctacagtgccgagcacagcgccttggctcttccagtgtgaggcccggcttatatcactactgcttcatggcaccatacacgcacttcacgcaggctttggctgatgccagcctgaggaacatggtacaggcggaccaggagcacgatgcctcagggtggtttgatgtactccagaaagtgtctgctcagttgaagacgaaccttacaagtgtcacaaagaaccgtgcagataagaacgctattcataaccacattaggttatttgagcctcttgttataaaagcattgaagcagtacaccacgacaacatcagtacaactgcagaagcaggttttggatttgctggcacagctggttcagctacgggtcaattactgtctactggattcagatcaggtgttcatcgggtttgtgctgaagcagtttgagtacattgaagtgggccagttcagggaatcagaggcaattattccaaatatatttttcttcctggtactattatcttatgagcgctaccattcaaaacagatcattggaattcctaaaatcatccagctgtgtgatggcatcatggccagtggaaggaaggctgtcacacatgctattcctgcgctgcagcccattgtccatgacctctttgtgttaagaggaacaaataaagctgatgcagggaaagagcttgaaacccagaaggaggtggtggtctcaatgctgttacgactcatccagtaccatcaggtgctagagatgttcatcctcgtcctgcagcagtgccacaaagagaatgaggacaagtggaaacgctctctcggcaggtcgcagacatcatcctgcccatgttagccaagcagcagatgcatattgactctcatgaagcccttggagtattaaataccttgtttgagattttggctccttcctccctacgtcctgtggacatgcttttgcggagtatgttcatcactccaagcacaatggcatctgtaagcactgtgcagctgtggatatctggaatcctagccattctgagggttctcatttcccagtcaaccgaagacattgttctttctcgtattcaggagctctccttctctccatatttaatttcctgtccagtaattaacaggttaagggatggagacagtaatccaacactaggagaacgcagtagagggaaacaagtaaagaatttgccagaagatacattctcaaggtttctcttacagctggttggtattcttctggaagacattgttacaaaacagctcaaagtggacatgagtgaacagcagcatacattctattgccaagagctcggcacactgctcatgtgtctgatccacatattcaaatctggaatgttccggagaatcacagccgctgccactagactcttcaccagtgatggctgtgaaggcagcttctatactctagatagcctgaatgcacgggtgcgagccatggtgcccacacacccagctctggtactgctctggtgtcagatcctactgctcatcaaccacactgaccaccgatggtgggccgaggtgcagcagacgcccaagagacacagtctgtcctgcacgaagtcactaaacccccagatatctgctgaagaggattctggctcagcagctcagcttggaatgtgcaatagagaaatagtacgaagaggggcccttattctcttctgtgattatgtctgtcagaatctccatgactcagaacacttaacatggctcattgtgaatcacattcaagatctgatcagcttgtcccacgagcctccagttcaagactttattagtgccattcatcgtaattctgcagctagtggtctttttatccaggcaattcagtctcgctgtgaaaatctttcaactccaaccactctgaagaaaacacttcagtgcttggaaggcatccatctcagccagtctggtgctgtgctcacactgtatgtggacaggctactgggcacccctttccgtgcgctggctcgcatggtcgacaccctggcctgtcgccgagtagaaatgcttttggctgcaaatttacagagcagcatggcccagttgccagaggaggaactgaacagaatccaggaacacctccagaacactgggcttgcacaaagacaccaaaggctctattcactgctggacagattccgactctctactgtgcaggactcacttagccccttgcccccagtcacttcccaccctctggatggggatgggcacacatccctggaaacagtgaatccggacaaagactggtacctccagcttgtcagatcccagtgttggaccaggtcagattctgcactgctggaaggtgcagagctggtgaaccgtatccctgctgaagatatgagtgacttcatgatgagctcggagttcaacctaagcctttttgctccctgcttaagccttggcatgagcgagattgcagggagccaaaagagtcccctttttgaagcggctcgtagggtgactctggaccgggtgaccaatgtggttcagcagctgcctgcagtccatcaagtcttccagcctttcctgcctacagaacccacagcctactggagcaagctgaatgatctctttggtgataccacatcataccagtctctgaccacacttgcccgtgccctggcacagtacctggtggtgctctccaaagtgcctgctcctttgcaccttcctcctgagaaggaggggcacacggtgaagtttgtggtaatgacacttgaggccctgtcatggcatttgatccatgagcagatcccactgagtctggacctccaagccggcctagactgctgctgcctggcactgcaggtgcctggcctctggggggtgctgtcctccccagagtacgtgactcatacttgctcccttatccactgtgtgcgattcatcctggaagccattgcagtacaacctggagaccaacttcttggtccggaaagcaggtcacatactccaagggctgtcagaaaggaggaagtagactcagatatacaaaacctcagtcacatcacttcggcctgcgagatggtggcagacatggtggaatccctgcagtcggtgctggccctgggccacaagaggaacagcaccctaccttcatttctcacagctgtgctgaagaacattgttgtcagtctggcccgcctccccctcgttaacagctatactcgtgtgcctcctctggtatggaaactcgggtggtcacccaagcctggaggggatttcggcacagtgtttcctgagatccctgtagagttcctccaggagaaggaggtcctcaaggagttcatctaccgcatcaacaccctagggtggaccagtcgtactcaattcgaagaaacttgggccaccctccttggtgtcctggtgactcagcccttggtgatggaacaggaagagagcccaccagaggaagacaccgaaaggacccagatccacgtcctggctgtacaggccatcacctctctagtgctcagcgcaatggctgtgcctgtggctggcaatccagctgtaagctgcttggagcaacagccccggaacaagccactgaaggctctcgataccagatttggaagaaagttgagcatgatcagagggattgtagaacaagaaatccaagagatggtttcccaaagagagaatactgccactcatcattctcaccaggcatgggatcctgtcccttctctgttaccagctactacaggtgctcttatcagccatgacaagctgctgctgcagatcaactcagagcgggagccaggcaacatgagctacaagctgggccaggtgtccatacactccgtgtggctggggaacaacatcacacccctgagagaggaggaatgggatgaggaggaggaggaagaagcggatgcccctgcgccaacatcaccacctgtgtctccagtcaattccagaaaacaccgtgctggggttgatattcactcctgttcgcagtttctgcttgaattatacagccgttggatcctgccatccagtgcagccagaaggacccctgtcatcctgatcagtgaagtggttcgatctcttcttgtggtgtcagacttattcactgacgtaccccagtttgaaatgatgtatctgacgctgacagaactacggagagtgcacccttcagaagatgagatcctcattcaatacctggtgcctgccacctgtaaggcagctgctgttcttggaatggacaaaactgtggcagagccggtcagccgcctactggagagcacactcaggagcacccacctgcccagccagatcggagccctgcatggcatcctctatgtgttggagtgtgacctcttggatgacactgtaaagcagctcattccagttgttagtgactatctgctgtccaacctcaaaggaatagcccactgcgtgaacattcacagccagcagcatgtgctggtgatgtgtgccactgcattctacctgatggaaaactaccctctggatgtggggccagaattctcagcatctgtgatacagatgtgtggagtaatgctgtctggaagtgaggagtccaccccctccgtcatttaccactgtgccctccggggtctggaacggctcctgctgtctgagcagctctctcggctagacacggagtccttggtcaagctaagtgtggacagagtgaatgtacaaagcccacacagggccatggcagccctaggcctgatgcttacctgcatgtacacaggaaaggaaaaagccagtccaggcagagcttctgaccccagccctgctacccctgacagcgagtctgtgattgtagctatggagcgagtgtctgtgctctttgacaggatccgcaagggatttccctgtgaagccagggtcgtggcaaggatcctgcctcagtttctagatgacttctttccacctcaagatgtcatgaacaaagtcattggagagttcctgtccaaccagcagccatacccacagttcatggccactgtagtatacaaggtttttcagactctgcacagtgctgggcagtcatccatggtccgggactgggttatgctgtctctgtccaacttcacacaaagaactccagttgccatggccatgtggagcctctcctgcttaccttgtcagtgcatctaccagcccatgggtttctgcaatccttccacacgtcatcagcaggatgggcaaactagagcaggtggatgtgaaccttttctgcctggttgccacagacttctacagacaccagatagaggaggaattcgaccgcagggctttccagtctgtgtttgaggtggtggcagcaccaggaagtccataccacaggctgcttgcttgtttgcaaaatgttcacaaggtcaccgcctgctgagtagtacctgtggaacaagaggctgagaggaggcaactgctgtggctacagcctccaggggcctgcaccaagcttctgctaaggctgccttggacgtgcaggcttccacttgtgtcaagtggacagccaggcaatggcaggagtgctttgcaatgagagctatgcagggaacatgcactatgttggggttgagcctgagtcctgggtcctggcatcactgcagctggtggcagtgctaggttgaccaggtgtttgtctttttcttagtgttgccctggccatagttgccaggttgcagctgccctggtatgtggaacagaatccgagctcttgtaagatggttctgagcccccctgtcccactgggctggagagctccctcccacatttacccagcaggtgtacctgccacaccagtgtctggacacaaagtgaatggtgtgggggctgggaactgggactgccaggtgtccagcatcattttccctttctctgttttcttctcaggagttaaaatttaattatatcagtaaagagattaattttaatgtaaaa

(19) United States
(12) Patent Application Publication
(10) Pub. No.: US 2007/0044162 A1
(43) Pub. Date: Feb. 22, 2007

(54) Transgenic rat as animal model for human huntingdon's disease

(76) Inventors: Olaf Riess, Tuebingen (DE); Stephan Von Hoersten, Wedemark (DE)

Correspondence Address: WILLIAM COLLARD;COLLARD & ROE, P.C., 1077 NORTHERN BOULEVARD, ROSLYN, NY, 11576, US

(21) Appl. No.: 10/514,512

(22) PCT Filed: May 14, 2003
(86) PCT No.: PCT/DE03/01546

§ 371 Date: Jun. 10, 2005

(30) Foreign Application Priority Data
May 14, 2002 (DE) 102 21 344.5

(51) Int. Cl. A61K 48/00; A01K 67/027; C07H 21/04; C12N 5/06; C12N 5/08

(52) U.S. Cl. 800/14; 424/93.21; 435/366; 435/353; 435/320.1; 536/23.2

(57) ABSTRACT

Huntington's Disease (HD) is an autosomal-dominant inherited progressive neurodegenerative disease from the group of CAG repeat/polyglutamine diseases and is characterized by a triad of psychiatric alterations, dementia and motor dysfunction. On a sub-cellular level, a mutation with extended CAG tri-nucleotide repeats has been identified as the cause of HD. The therapeutic effects of certain substances can be tested on neurotoxically-induced or transgenic animal models with expanded CAG-repeats. In the present invention, transgenic rats were generated and characterized for human HD. Said rat model for human HD and other diseases of the CNS carries 51 CAG repeats under the control of a rat promoter and has a slow progressive neurological phenotype, closely reflecting human HD syndrome. The comparability of the rat model in relation to human HD is characterized by neuropathological, neuroradiological and neurochemical modifications accompanied by typical behavioral symptoms.

### FIELD OF THE INVENTION

The invention relates to a nucleic acid construct, vectors and cells containing this nucleic acid construct, as well as the use of these means for the generation of a transgenic non-human mammal, especially a rat as an animal model for the human Huntington's disease.

### BACKGROUND OF THE INVENTION

Chorea Huntington (Huntington's disease, HD) is an autosomal dominant hereditary neurodegenerative disorder from the group of the “CAG-repeat”/polyglutamine-disorders. The course is typically slowly progressive spanning a period of 15-20 years. The onset is in the middle age of life and is characterized initially by emotional disturbances and psychiatric abnormalities (depression, addiction, psychosis). Upon progression of the disease, dementia along with hypo- or hyperkinetic (choreatic) motor dysfunction becomes apparent. On the cellular level at this point of time, on a pathophysiologal level a selective degeneration of striatal and cortical neurons is present, which in the final stage leads to an enlargement of the lateral ventricles of the brain. On a sub-cellular level, a mutation (gene IT15, chromosome 4) with elongation of CAG-tri-nucleotide repeats has been identified as the pathogenetic cause of HD. Tri-nucleotide expansions of more than n>37 repeats lead to an HD phenotype, wherein at increasing numbers of repeats the course of the disease is more severe and the onset of the disease occurs earlier. Intranuclear aggregates of huntingtin, heat shock proteins, and ubiquitin in striatal neurons are pathognomonic for HD. A number of animal models have been generated so far, either by injection of neurotoxics as well as by genetic manipulation of mice and drosophila. Among those, the R6/2 mouse model of HD presently is most frequently used, though in this model the course of the disease is protracted and diabetes mellitus is apparent as co-morbidity. Due to the fulminating course of the disease in R6/2 mice in comparison to man, studies on the course (e.g. radiologically by MRT or PET) as well as therapeutic studies (e.g. neurosurgically by transplantation of stem cells) are very possible only within limitations.

Choreiform Movement Disorders in Humans

The group of choreiform movement disorders is generally caused by different neuropathological disturbances, which have an impact on the highly vulnerable basal ganglia. Causes are many forms of vascular, infectious, traumatic, neo-, and para-neoplastic, metabolic or immunological but especially neurodegenerative diseases having in part hereditary components (Table 1).

TABLE 1
Hereditary and secondary causes
of choreiform movement disorders

| hereditary | non-hereditary |
|---|---|
| Huntington's disease | Sydenham's Chorea, other |
| Huntington's disease like chorea | infection - associated diseases |
| Atrophia of tractus | Chorea in pregnancy |
| dentatorubrospinalis | (possibly contraceptives) |
| Spinocerebellar ataxias, | vascular |
| mainly SCA3 | |
| Paroxysmal chorea athetosia | Drugs: Neuroleptics, L-Dopa, steroids |
| Neuroacanthocytosis | Metabolic |
| M. Wilson | Trauma |
| Benign familial chorea | Neoplasties and paraneoplastics |
| Dystonia with torsions | Postoperative brain damage |
| Lesh-Nyhan-Syndrome | Perinatal hypoxic brain damage |
| Hallervorden-Spatz-Disease | Metabolic disorders in childhood |

Genetically Caused Choreiform Movement Disorders

As a differential diagnosis after exclusion of secondary causes of choreiform movement disorders and other genetically caused diseases, respectively, wherein chorea is an accompanying symptom, especially HD (chorea Huntington, chorea major, morbus Huntington) has to be taken into consideration in differential diagnostics. Especially recently, novel molecular genetic techniques could demonstrate that apart from Huntington's disease there are other, clinically hardly to differentiate hereditary diseases (Huntington-like diseases) (Table 2).

TABLE 2
Gene loci of hereditary choreiform movement disorders

| | | Chromo- | | |
|---|---|---|---|---|
| | Mode of | somal | | |
| | inheri- | locali- | | Type of |
| Disease | tance | zation | Gene locus | mutation |
| Huntington's | AD | 4p16.3 | IT-15 | CAG- |
| disease | | | (Huntingtin) | expansion |
| Huntington- | AD | 20p | Prion gene | insertion |
| like chorea | | | | |
| (HD-L1) | | | | |
| Huntington - | AD | 16q23 | Junctophilin- | CAG- |
| like chorea | | | 3 | Expansion |
| (HD-L2) | | | | |
| Huntington - | AR | 4p15.3 | Unknown | |
| like chorea | | | | |
| Benign chorea | AD | 14q | Unknown | |
| Chorea | AR | 9q21- | CHAC-Gene | Point |
| acanthocytosis | | q22 | | mutation, |
| | | | | deletion, |
| | | | | insertion, |
| | | | | duplication |
| Chorea | XR | Xp21.2 | XK-Gene | Point |
| acanthocytosis | | | | mutation |
| (McLeod's | | | | |
| syndrome) | | | | |
| Choreatic | AD | 2q33- | unknown | |
| athetosis, | | q35 | | |
| paroxysmal, | | | | |
| dystonic, | | | | |
| non-kinetogenic | | | | |
| (Mount- | | | | |
| Reback, DYT8) | | | | |
| Choreatic | AD | 1p | unknown | |
| athetosis, | | | | |
| paroxysmal | | | | |
| with episodic | | | | |
| ataxia and | | | | |
| spastics (DYT9) | | | | |
| Choreatic | AD | 16p11.2- | unknown | |
| athetosis, | | q12.1 | | |
| paroxysmal | | | | |
| kinetogenic | | | | |
| (DYT10) | | | | |
| Choreatic | AD | 16p12- | unknown | |
| athetosis, | | q12 | | |
| paroxysmal | | | | |
| with infantile | | | | |
| fever spasms | | | | |
| (ICCA syndrome) | | | | |

Abbreviations: AD: autosomal dominant, AR: autosomal recessive, XR: X-chromosomal recessive.

Huntington's Disease (Chorea Huntington, Chorea Majors Huntington's Disease, HD)

Clinical Symptoms of HD

The clinical symptoms of HD can be summarized as the “classic” triad of movement disorder, organic changes of the personality (psychiatric symptoms), and cognitive decline (dementia). These symptoms become apparent in most affected persons between the 35^{th} and 45^{th} year of life, but 5-10% manifest before the 20th year of life. Quite often the disease starts with psychiatric symptoms, mainly depression and cognitive decline, which precede the other symptoms by several years.

Motor Dysfunction in HD

HD is named commonly still “Huntington's chorea”, “chorea major”, or “inherited choreiform dancing”. This expression takes into account the main symptom, the choreiform (dancing-like) movement disorder, which in the greater number of patients represents the most prominent feature of the disease. Apart from this typical choreiform symptoms, in some patients other motor dysfunctions are found. Often dystonias, especially in the distal regions of the extremities or the neck and jugular region cause unnatural postures. However, in principle all forms of hyperkinetic symptoms, such as (hemi)ballism, asterixis, myoclonus, and tremor can occur.

A specific form of the disease is the so-called akinetic-rigid juvenile form of the disease, or “Westphal-variant” (3-10%). It predominantly occurs in young patients after paternal inheritance having high numbers of CAG repeats (see “Genetics”). Clinically, a Parkinson-like symptomatology with pronounced Brady- or akinesia, high stiffness of the muscles, and a rapid progression makes an impression. In these patients, also development of dementia typically appears quicker. The dystonia component is more pronounced compared to the hyperkinetic form and patients are massively slowed down and nicked forward. Disturbance of swallowing causes very early strongly pronounced saliva efflux. Also, mixed forms with simultaneous hyperkinesia as well as dystonic or rigid increase of the tonus are observed in younger patients often, which can then blur diagnosis.

Psychic Disturbances in HD

Early psychiatric symptoms of the disease are depressive moods. In most patients a strong non-perception up to abnegation of signs of the disease exist. Anyhow, the depressed mood often is expressed by a somatization, especially pain syndromes are frequent. The kind of changes in personality and especially of the affective disorder often depends on the healthy primary personality of the patient. This goes along with delusional misjudgements, which can manifest itself as delusion of jealousy or paranoid disorder. Real schizophrenic symptoms such as visual or acoustic halluzinations seem to be rather rare in early stages except for the Westphal variant or their mixed forms but appear with progression of the disease.

Cognitive Decline in HD

The progressive development of dementia manifests itself early as a loss of professional performance. The reduction in intellectual capacities initially affects the concentration ability and the performance of memory and remembering by the acute memory. A pronounced slowing of thinking and disorder of perception, as it is typical for sub-cortical dementia, accompanied by a pronounced fixation to certain contents of thinking, are frequent. The integration of different cognitive functions, the constructive performance and especially the verbal working memory are affected, but in contrast e.g. to the dementia of the Alzheimer's type, disorders of speech, such as aphasia or apraxia are rarely found in early stages. The cognitive decline in M. Huntington is additionally connected with disturbances of motivation, emotion, and with pronounced changes of personality.

Neuropathology in HD

Neuropathologically, in patients with HD a degeneration of nerve cells in the CNS is found, primarily in the caudate nucleus, subthalamic nucleus, and putamen. Medium-sized neurons containing gamma-aminobutyric acid and enkephalin or gamma-aminobutyric acid and substance P as neurotransmitters are most severely affected (Martin und Gusella 1986). In the progressed stage, often the whole brain is atrophic, which is accompanied macroscopically by an enlargement of sulci, shrinkage of gyri and a reduction of the total brain mass. The massively compromised coordination of movements also leads to problems in swallowing of food, causing cachexia along with pneumonia in a proportion of patients. This is also the main cause of death (33%) in HD patients, followed by heart and circulatory diseases (24%). The average age of death in HD is 57 years.

Continuative Diagnostics in HD

The clinical-instrumental diagnostic is composed of imaging techniques, neuropsychological testing, and special precision motor examinations. To these, electrophysiological examinations, especially somatosensorily evoked potentials (SSEPs) and so-called “long-loop-reflexes”, which played an important role in the early diagnostics before introduction of genetic tests are added.

Among the imaging techniques, cranial computer tomography (CCT) is best examined. Along with manifold other markers of atrophia, measurement of the width of the ventricular cornu anterior in comparison to the width of the cornu posterior at the level of the third ventricle and the ratio of the width of cornu anterior to total width of the temporal brain at this level, respectively, have proved efficient. In the end, both parameters score the atrophia of the caput of the nucleus caudatus, which as a part of the basal ganglia very early in HD degenerates and the size of which can be directly determined due to its protuberation into the cornu anteriores of the lateral ventricles (so-called “Huckmann's number”). In later stages this selective atrophia of the basal ganglia gives way to a generalized neurodegeneration, which also pertains to cortical proportions, the examination is therefore little specific.

For the experienced investigator, also measurement of the lateral ventricles in transcranial duplex sonography allows to identify subcortical atrophia as well as disease progression. Positron emission tomography (PET) (see also FDG-PET, Kuwert et al. 1990) provides insight on perfusion and glucose metabolism of the basal ganglia and can be used as an early diagnostic marker, typically, however, PET remains for dedicated questions. As an instrumental test series on fine motor functions, a testing apparatus “Motorische Leistungsserie” (Schoppe 1974) can be used.

Neuropsychological testing can be obtained by an elaborate testing apparatus registering the performance of memory, especially in the field of verbal memory but also in visual-constructive performances. Apart from dysfunction of memory, also the active part of the “Hamburg-Wechsler-Test-of-Intelligence” (HAWIE) is said to be compromised early on (Lyle and Gottesman 1977). For a quick clinical detection of cognitive functions word-finding tests (construction of as many words as possible out of single letters), Stroop-tests (e.g. test of interference: the color of a word, describing another color, has to be named), and the “inter-digit-span-test (numbers must be assigned to certain symbols) have proved efficient and may also be used for monitoring disease progression.

Incidence and Genetics of HD

Morbus Huntington (HD) is an autosomal dominant hereditary neurodegenerative disease. In Central Europeans HD has an incidence of 4-8 afflicted persons per 100,000 inhabitants. The disease in Japanese (4:1×10⁶), Finns (5:1×10⁶), and Africans (6:10×10⁶) is more rare (overview in Harper, 1992). New mutations are extremely rare and most of the time due to missing clinical data or early death of parents. Maximal 3% of patients with ascertained HD become diseased due a new mutation.

Phenotype-Genotype-Correlation in Patients with HD

There exists an inverse correlation between appearance of first symptoms of HD and repeat length. Patients that become affected after the 60th year of life typically carry less than 45 CAG-units on the mutated allele. In contrast, patients with 55 CAG or more units mostly become diseased before the age of 30 years. The expansion of repeat length, but not the normal allele, determines up to 60% of the variability of age-of-onset in HD patients. Other genetic factors such as the glutamate receptor R6 (Rubinsztein et al. 1997), the transactivator protein CA150 (Holbert et al. 2001) or the age-of-onset of parents have an impact on the age-of-onset. However, these analyses do not have relevance for the clinical practice and predictive analyses, respectively.

Juvenile HD (Westphal-Variant)

Up to 10% of HD-patients become diseased before the age of 20 years (for clinical symptomatology see previous section). In some cases children show first symptoms already before their parents, which provides the impression of a new mutation or the omission of a generation. More than 80% of juvenile patients have inherited from their father the mutation (CAG)ₙ, which usually has expanded further during paternal transmission. All juvenile patients had more than 45 CAG repeat units. Children showing HD-symptoms already before the age of 10 years carried more than 75 CAG repeat units. An analysis of repeat-length in sperm cells of affected individuals revealed a clear somatic instability, i.e. the major portion of male germ cells carry a longer allele than detectable in the peripheral blood. The degree of somatic instability of the CAG-repeat in sperm is directly correlated to the degree of repeat expansions during transmission to offspring (Telenius et al. 1995). Summarizingly, it can be said that the clinical phenomenon of anticipation, i.e. an earlier onset of symptoms with a more rapid progress in the offspring of affected parents is caused by an expansion of CAG repeat units, predominantly (but not exclusively) during paternal transmission and that a juvenile onset of the disease is caused by very long expansions.

The Intermediary Range of 30-40 Repeat Units

The range of CAG units, which could be demonstrated in healthy as well as in proven affected individuals shows an overlap in only a few persons. Seven HD patients world-wide have been described exhibiting only 36 CAG repeats, whereas a few individuals carrying 36-39 CAG units show even in high age (more than 90 years) no symptoms of the disease (Rubinsztein et al. 1996).

Diagnostic Procedures in HD

Traditionally, diagnosis of HD was clinically based on a positive family anamnesis, progressive voluntary and involuntary motor dysfunction and/or psychiatric symptoms. The proof of atrophy of the C. caudatus and putamen in CT or MRT confirmed the diagnosis. Using positron-emission-tomography (PET) a reduced glucose metabolism in the nucleus caudatus can be visualized before cell loss can be determined in CT or MRT. Despite these criteria false positive and false negative wrong diagnosis are frequent at about 10% each. A definite exclusion or confirmation of HD is even in the early stage only possible by DNA analysis. A CAG repeat number of more than 38 repeats in the Huntingtin gene is considered firm proof of HD.

Molecular Genetic Causes of HD

In 1983 the defect causing HD was localized on the short arm of human chromosome 4 (subregion 4p16.3). Only 10 years later the Huntingtin gene, originally termed gene IT15, was isolated (The Huntington's disease collaborative research group 1993). The molecular defect was identified in form of an expanded (CAG)_{>38} repeat in the coding region of the gene (Kremer et al., 1994, Zühlke et al., 1993). This elongated CAG-repeat, but not the normal alleles behaves instable during meiosis, i.e. during germ cell development it can be transmitted to the next generation in a shortened (rare) or further elongated form (meiotic instability), which is the cause for the clinical phenomenon of “anticipation” (Telenius et al., 1995). Especially in massively expanded alleles also mitotic instability is found, in particular in the basal ganglia and in the cerebral cortex (Telenius et al. 1994).

Function of the Huntingtin-Protein in HD

The (CAG)ₙ repeat of the huntingtin gene codes for the amino acid “glutamine”, so that HD is also spoken of as a polyglutamine disease. The DNA sequence analysis of the huntingtin gene did not yield any hint for a homology to other known genes and, accordingly, to the function of the gene product. Individuals lacking one of the two chromosomal portions carrying the huntingtin gene do not develop symptoms typical for HD. Presently, therefore, it is thought that the polyglutamine expansion generates a new property of the protein, possibly to a non-specific binding of the elongated polyglutamine domain to another protein. The normal huntingtin possibly plays a functional role in stimulating transcription of the brain-derived neurotrophic growth factor, BDNF (Zuccato et al. 2001). Mutated huntingtin stimulates the expression of BDNF only insufficiently.

Pathogenetic Characteristics of HD)

The putative structure of the elongated polyglutamine chains early on allowed to conclude that huntingtin possibly aggregates with itself and other proteins, respectively (Perutz 1996). Cell culture experiments (Scherzinger et al., 1997) and immunohistological analysis on transgenic animals (Davies et al 1997) as well as later on brains of deceased patients (DiFiglia et al., 1997) finally yielded the proof for the theory of Perutz by providing evidence for the existence of huntingtin-positive aggregates in the cell nuclei of neurons. Thereafter, these neuronal nuclear inclusion bodies could be detected in almost all other polyglutamine diseases, such as SBMA, DRPLA and SCA 1, 2, 3, as well as 7. Overexpression of heat shock proteins and the stimulation of heat shock proteins, respectively, by pharmaceuticals such as geldamycin reduces huntingtin aggregation (Sittler et al. 2001).

Presently Available Animal Models of HD

During the past 20 years a number of trials for the generation of animal models of HD have been undertaken. Initially, excitotoxin and 3-nitropropionic acid models have gained special importance, since they allow to replicate several of the histological features and some motor functional symptoms including alterations of gait and cognitive symptoms of HD (Borlongan et al., 1995; Brouillet et al., 1995; Brouillet et al., 1999). However, since these models are neurochemically induced, the symptoms are not really progressive. Therefore, these neurotoxic models can only be used within limits for therapeutic studies, when these aim at an prohibition or slowing down the course of HD.

It is therefore of importance that transgenic models of HD are generated. Transgenic models of HD in mice (Hodgson et al., 1999; Mangiarini et al., 1996; Reddy et al., 1998; Schilling et al., 1999; Shelbourne et al., 1999; Wheeler et al., 2000; Yamamoto et al., 2000) allow novel approaches for examining the causative mechanisms of their progressiveness (Li et al., 2000) as well as the pathogenetic causes of HD (Brouillet et al., 1995, 1999, 2000). The therapeutic effects of certain compounds in regard to the onset of disease and progress of HD can therefore be tested in animal models as well. All models have in common that the expanded CAG-repeat of patients has been transferred into the germ line of mice or drosophila (transgenic animals). So far, the most widely spread animal model (R6/2 mice) has been generated using a fragment of the huntingtin gene with more than 113 CAG repeats (Mangiarini et al 1996). The R6/2 transgenic mouse expresses the first exon of the human HD gene with 114-157 CAG repeating units (repeats) and develops a number of characteristic symptoms of HD, including progressive motor dysfunction (Mangiarini et al., 1996; Dunnett et al., 1998; Carter et al., 1999), neuro-pathologically presence of neuronal inclusion bodies (Davies et al., 1997). In addition, this mouse model has impaired learning capabilities (Lione et al. 1999) and reduced anxiety (File et al. 1998). Thus, numerous behavioural studies in principle demonstrate the comparability between pathology and symptoms in mice to the disease in humans.

However, R6/2 mice show a fast progress with a fulminating progressive phenotype. This course is rarely found in humans and only when children are already affected by the disease (so-called Westphal variant). Diabetes mellitus is frequently observed in young animals already (Carter et al., 1999). This rapid progress along with co-morbidity makes the determination of effectiveness of potential therapeutics and repair strategies (neuronal cell transplantation) for the treatment of symptoms of HD difficult.

Therapy and Studies on the Progress in Transgenic Animal Models of HD

Therapeutic effects of certain compounds on the progress of HD can be tested in transgenic animals having expanded CAG repeats, but this has not been published to a larger extend. An exception is found in experiments with Drosophila that demonstrate that overexpression of chaperons of the HSP70 gene family blocks death of neuron cells (Warrick et al., 1999). Using the R6/2 line, it could be shown that inhibition of caspase causes a delay in the progress of the disease (Ona et al. 1999). This effect can also be replicated using Minozyklin, a drug used for the treatment of acne vulgaris, which inhibits caspase-1 and 3 (Chen et al. 2000). Finally, it could be demonstrated in R6/2 mice that keeping these animals in a stimulating environment (environmental enrichment) leads to a clear delay of neurological symptoms (van Dellen et al. 2000, Hockly et al. 2002).

In principle, also the future treatment of patients, which are already affected is not without hope. If only the dysfunction of the aberrant protein, its degradation could be accelerated, and/or its transport into the nucleus, could be blocked, respectively, possibly clear therapeutic success could be expected. This conclusion has at least been supported by another animal model, in which the transgene can switched on and off inducibly (Yamamoto et al. 2000). Therein, it was demonstrated that in animals that initially already showed clear symptoms, the HD phenotype was reversible after switching off of the transgene and neuropathologic features like inclusion bodies regressed. Thus, the latter studies demonstrate the reversibility of the HD phenotype in principle.

Therefore, there is an urgent need for transgenic animal models for studies on chorea Huntington and for the search for suitable therapeutic measures.

### SUMMARY OF THE INVENTION

The present problems in therapeutic and progress studies in animal models of HD, the following facts can be summarized:
(1) neurochemically induced animal models show no progress; (2) the wide spread transgenic animal model of the R6/2 mouse exhibits a fulminating rapid progressive course; (3) the R6/2 mouse has diabetes as co-morbidity.

The problem to the present invention was, therefore, to supply a transgenic animal as a model for the neurodegenerative disease chorea Huntington, which reflects the progress of the disease in humans quite closely and especially the more slowly progress of the disease than in other known animal models. Other effects, which are not related to HD, which can be initiated e.g. by a certain co-morbidity, should possibly not occur.

This problem is solved by a special nucleic acid construct on the basis of the rat huntingtin gene (RHD10), described in the following in further detail, and related vectors and cells, which allow the generation of a transgenic mammal, namely especially a transgenic rat, which does not exhibit the disadvantages of the state of art. Surprisingly, it could be demonstrated that the transgenic rat according to the present invention better reflects the course of the disease in humans than the mouse models used so far, although in both cases they are rodents. Diabetes mellitus, present in mice, does not seem to occur. Further, other co-morbidities cannot be recognized. In comparison to the mouse, the course of the disease in the rat is clearly slowed down, allowing to better carry out studies on the course (over several slowly developing stages). A beneficial side effect of the rat lies in the fact that it is a little larger than the mouse, allowing to better perform imaging processes and surgery.

In the present invention, a rat model was developed for human HD, which exhibits a slowly progressive neurological phenotype and especially reflects the most frequent, late-manifesting and slowly progressive form of HD. This is the first functionable rat model of a human neurodegenerative disorder of the CNS, which is induced by a transgene and is under control of a rat promotor. This rat animal model is very likely to gain exceptional importance for long-term progress monitoring including behavioral testing and PET, for long term treatments as well as many other therapeutic approaches, like e.g. microsurgery and stem cell transplantation.

A direct comparability of the rat model to human HD shows in neuropathological (inclusion bodies in the striatum), neuroradiological (enlarged lateral ventricles, focal lesions in the striatum in MRT, reduced glucose utilization in PET) and neurochemical (tryptophane metabolism in CNS) alterations along with typical behavioral symptoms. The symptoms of behaviour reflect the course of alterations in humans: already at the age of two months the animals impress with emotional symptoms like e.g. reduced anxiety in the “elevated plus maze” and in the “social interaction test of anxiety” and reduced curiosity (exploration within the “holeboard test”). At the age of ten months, clear cognitive alterations appear, like e.g. increased “working memory errors” and “reference memory errors” in the “radial maze test” for spatial learning. In the age of 10-15 months at the latest, additional deficiencies in tests for motor coordination appear, i.e. in the “accellrod test”. From about the 16th month of age animals increasingly show cachexia and increased lethality. These HDtg rats therefore represent the worldwide first rat model for a human neurodegenerative disease of the CNS and exhibit a whole number of parallels to human HD. In summary, it is to be assumed that these animals provide a useful model for therapeutic studies of neurodegenerative diseases in general and especially for the “CAG-repeat-disorders”.

At first, the present invention compromises a nucleic acid construct that is used for the generation of transgenic mammals in animal model studies. The nucleic acid construct according to the present invention contains at least one carboxyterminally truncated sequence of the rat huntingtin gene (RDH10), at least 36, especially approximately 40, further preferably more than 50 CAG tri-nucleotide repeats and furthermore upstream at least an effective portion of a huntingtin gene-specific promotor.

The minimal requirements required for the construct therefore compromise a regulatory unit (promotor) as well as the actual gene as a carrier of the protein encoding information, which here consists of a CAG repeat-containing and supplemented rat huntingtin gene portion.

The promotor to be used firstly depends on the species of the animal to be generated, although promotors foreign to the species can be used as well, as far as these effect the desired regulation. In the first line, promotors of the human, rat or mouse are considered, the native rat huntingtin gene promotor or a functional fragment thereof is preferred. In principle, every promoter can be used for rodents transgenic for HD which is expressed in the brain. Examples are the prion gene promoter, the PDGF promoter and the human Huntington promoter.

As a further development of the invention, the nucleic acid construct is equipped such that the CAG tri-nucleotide repeats are present within human Huntington gene portion integrated into the construct, which for example was obtained from patient DNA. Obtaining the human gene portion having the CAG repeats can for example be performed by PCR reproduction with primers Hu 4 (ATGGCGACCCTGGAAAAGCTGATGAA) and Hu3-510 (GGGCGCCTGAGGCTGAGGCAGC).

Regularly, the nucleic acid construct downstream from the rat huntingtin gene will contain a polyadenylation sequence, which after transcription mediates a poly-A attachment to the mRNA and an increase in mRNA stability.

Substitution of single components of the construct provided in the example by those of corresponding function is possible. For example, it is possible as given above, to use another promoter (e.g. from the human, rat or mouse), a shorter or longer cDNA gene fragment and construct, respectively, or even a direct integration of complete human, rat or mouse cDNA sequences can be made. Finally, constructs can differ by use of different poly-A-signals (frequently SV40). However, the order of all parts is always fixed. The promotor is located on the 5′-end, at 3′ immediately flanked by the cDNA, the end of which is determined by a stop codon and the poly-A signal.

The sequence of the rat huntingtin gene (also termed IT-15), and the carboxyterminally truncated portion of RHD10 relevant herein, respectively, is described in Schmitt et al., 1995 (see refs), as well as published under GenBank Accession No. U 18650. Preferably, the CAG repeat containing N-terminal end of the incomplete rat huntingtin gene (RHD10) is substituted by human patient DNA, which contains at least 36 CAG repeats. Furthermore preferably, this is done by inserting a PCR fragment, which was generated from a chorea Huntington patient.

The present invention further compromises vectors and mammalian cells, except human embryonic cells, which contain the nucleic acid construct, and which are transfected with this, respectively.

The nucleic acid construct according to the present invention, the vectors and cells are then used for the generation of transgenic using methods known in the art for the generation of transgenic animals. Preferably, non-human transgenic mammals are generated, especially transgenic drosophila, mice and rats.

The transgenic rat generated is characterized in that it contains in the genome of its germ line cells and somatic cells an aberrant sequence of the Huntington gene (HD10), expanded by CAG repeat units, which was transferred into this animal or one of its predecessors. Preferably, the gene sequence comprises at least 36, especially about 40 CAG tri-nucleotide repeats, and further preferred more than 50 (51 in the example).

The transgenic rat generated will serve as a model animal for performing studies on the course of the disease Chorea Huntington, for the development of therapeutic and/or prophylactic agents against this disease and comparable diseases, for the investigation of therapeutic concepts, for performing microsurgical surgery, for stem cell transplantations or for gene therapeutic treatments or antisense treatments.

In the following the present invention will be explained further by way of example and by way of figures.

### DESCRIPTION OF THE INVENTION

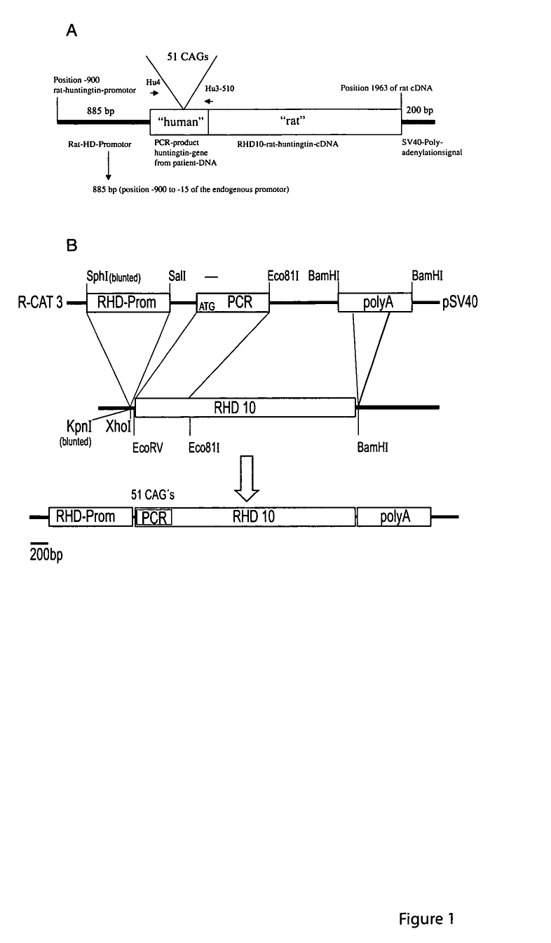

FIG. 1 in A shows a general overview on the genetic construct “RHD/Prom51A” for HDtg rats. The complete clone of the coding rat cDNA would span 9333 bp. For the transgenic animals only a part of the cDNA (1-1963 bp) out of the RHD10 construct was taken (Schmitt et al. 1995). The native rat huntingtin promoter (885 bp) (Holzmann et al. 1998) was taken as the promotor. FIG. 1B provides another survey on the construct. The first 154 bp of an incomplete rat huntingtin cDNA (RHD10) (Schmitt et al, 1995) were replaced by the PCR product of the allele from a diseased HD patient. The cDNA is under control of a 885 bp fragment of the rat HD promoter (positions −900 to −15 bp) (Holzmann et al., 1998). Finally, a 200 bp fragment having a SV40 polyadenylation signal is added downstream (3′), in total yielding the RHD/Prom51A construct.

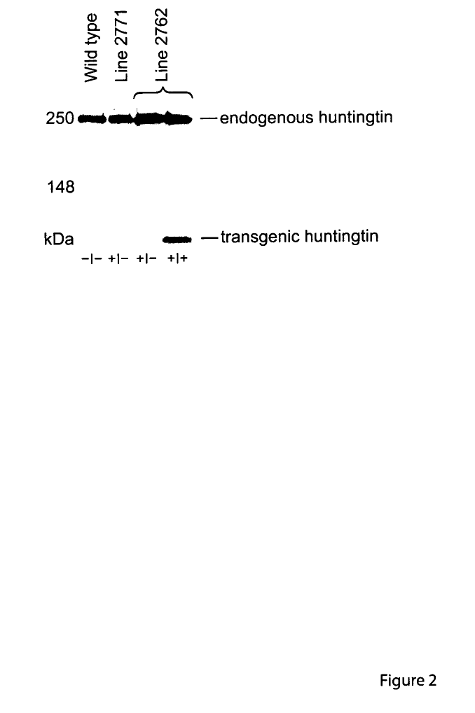

FIG. 2 provides a Western blot analysis (Schmidt et al. 1998) of brain tissue of wild-type and HDtg rats of line 2771 (heterozygous animal) and 2762 (heterozygous and homozygous animal). The polyclonal anti-huntingtin antibody 675 was used. There is shown a 75 kD reaction product proving the expression of the transgene on a lower level than the endogenous protein. Homozygous rats express about double the amount of the transgenic protein in comparison to heterozygous animals.

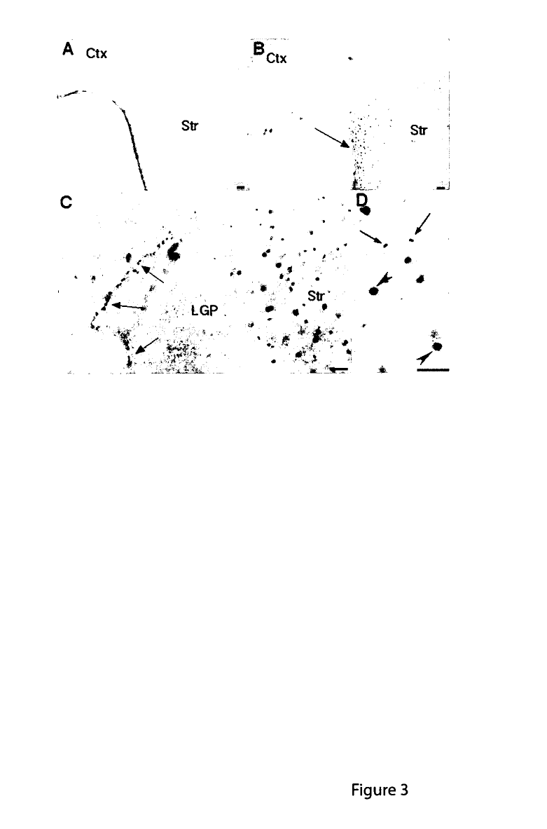

FIG. 3 shows neuropathological changes in frontal histological sections through the striatum of HDtg rats in the form of nuclear inclusion bodies and neurophilic aggregates. The black and white photographies A and B show microscopic fields of vision of wild-type (A) and HDtg (B) rat brains at the age of 14 months at low magnification. Immunoreactivity of EM48 immunoreactive is particularly enriched in the ventral part of striatum (Str) in immediate vicinity of the lateral ventricles (arrow) in the HDtg rat brain. Ctx: cortex. Scale bar: 50 μm. (C) In the nucleus caudatus of the striatum of HDtg rats, many nuclear aggregates and small neurophilic aggregates are found. Neuropilic aggregates (arrows) are also found in the lateral globus pallidus (LGP). Scale bar: 25 μm. (D) High magnification from the striatum of HDtg rats with both EM48-positive nuclear aggregates (arrowheads) and small neuropilic aggregates (arrows). Scale bar: 10 μm.

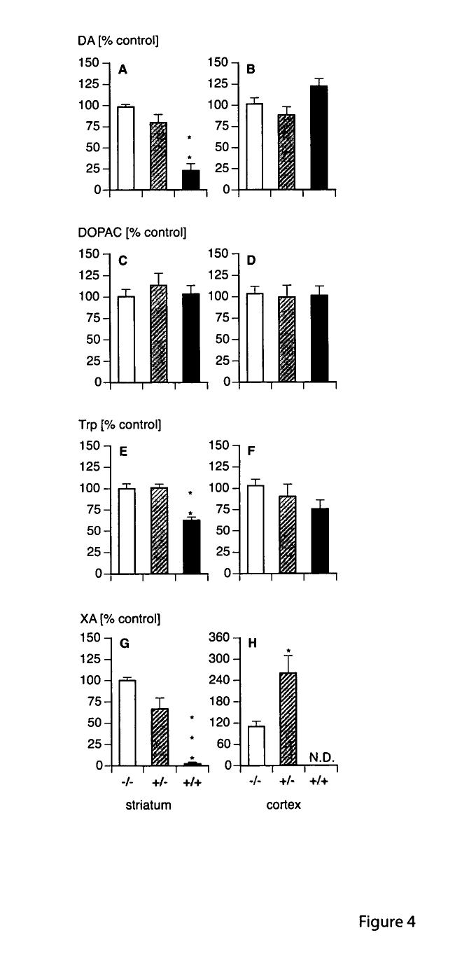

FIG. 4 shows specific differences in tissue concentrations of dopamine and kynurenic acid in single brain regions of the HDtg rats. The levels of dopamine (A, B), DOPAC (C, D), tryptophane (E, F) and xanthurenic acid (G, H) in striatum (A, C, E, G) or parietal cortex (B, D, F, H) in wild-type (−/−) or homo-(+/+) and heterozygous (+/−) rats, respectively, are provided. Asterisks indicate significant differences between wild-type (−/−) control rats and hetero- or homozygous HDtg rats (*p<0.05, **p<0.001, ***p<0.0001). Using neurochemical analyses, these studies show that the HDtg rats exhibit changes comparable to the human HD in humans.

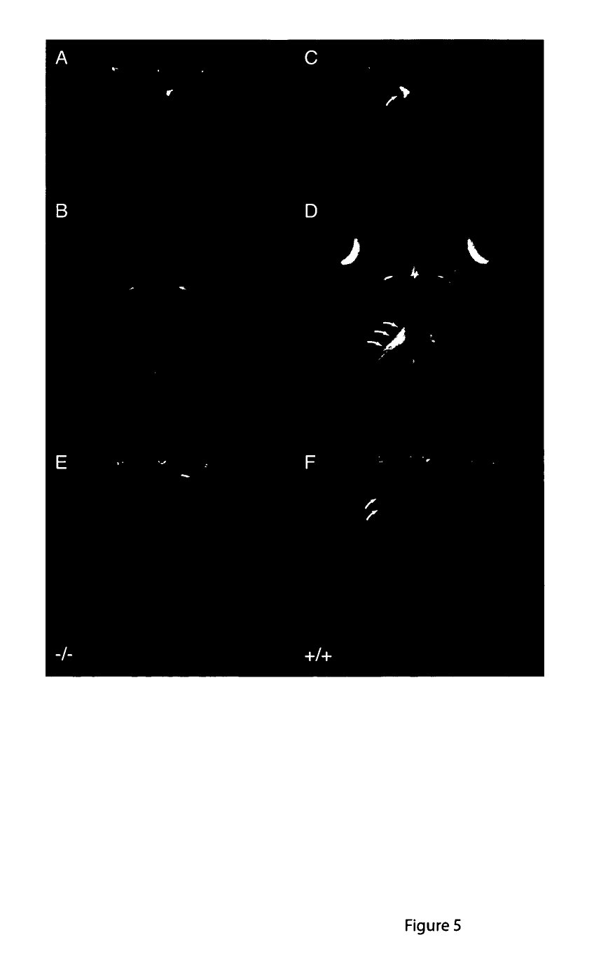

FIG. 5 shows neuroradiological changes in black/white copies of magnetic resonance tomography (MRT) of the brains of HDtg rats in the form of focal lesions in the striatum and in the form of enlarged lateral ventricles. (A-D) MRT scans of lateral ventricles in coronal (frontal) (A) and sagittal (B) projection of wild-type (A, B) and HDtg (C, D) rat brains. (E-F) MRT scans of the striatum at coronal level of a wild-type (E) and a HDtg (F) animal at the age of 8 months. Note the enlargement of the lateral ventricles (arrows in C and D) and the focal lesions in the striatum (arrows in F). These studies show by making use of the neuroradiological method MRT that the HDtg rats in comparison to human HD exhibit similar alterations.

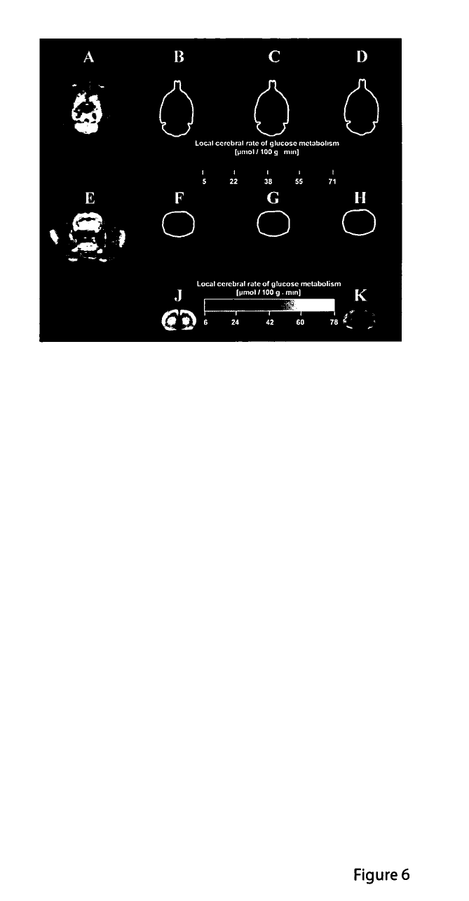

FIG. 6 shows changes in glucose utilization in high resolution [¹⁸F] FDG small animal PET in HDtg rats. The figure is composed of black and white converted representative images (originals in color) from [¹⁸F] FDG small animal PET in horizontal (B-D) and coronal (F-H) level sections together with individual MRT scans (A, E) and ex vivo autoradiographies (J, K). MRT scans (A, E) of a wild-type control animal are registered in parallel with respective [¹⁸F] FDG-PET images (B, F). Planes are cutting the caudato-putamen complex level of the brain. Sections for autoradiography (J, K) are taken from the identical animals as the [¹⁸F]FDG-PET images (B, F, D, H). Measuring range (regions of interest) within the [¹⁸F]FDG-PET images are defined using the corresponding MRT scans (clarified by the white line). The local rate of glucose metabolism (lCMR_{Glu}) are quantified absolutely (see black/white scale). The high accumulation of activity in the caudato-putamen region is clearly visible in [¹⁸F]FDG-PET images (F, G, H) and in autoradiographies (J, K). Homozygous transgenic rats exhibit significantly (p<0.05) lowered lCMR_{Glu} values compared to wild-type control animals, both in [¹⁸F]FDG-PET (34.54±18.52 vs. 54.98±15.53) and in ex vivo autoradiographies (43.54±6.77 vs. 63.02±8.24). Using the neuroradiological method PET these studies show that the HDtg rats in comparison to human HD exhibit similar disturbances in the glucose utilization. This approach furthermore demonstrates that it is possible in HDtg rats to carry out repeated PET progress analyses which are not possible in the mouse.

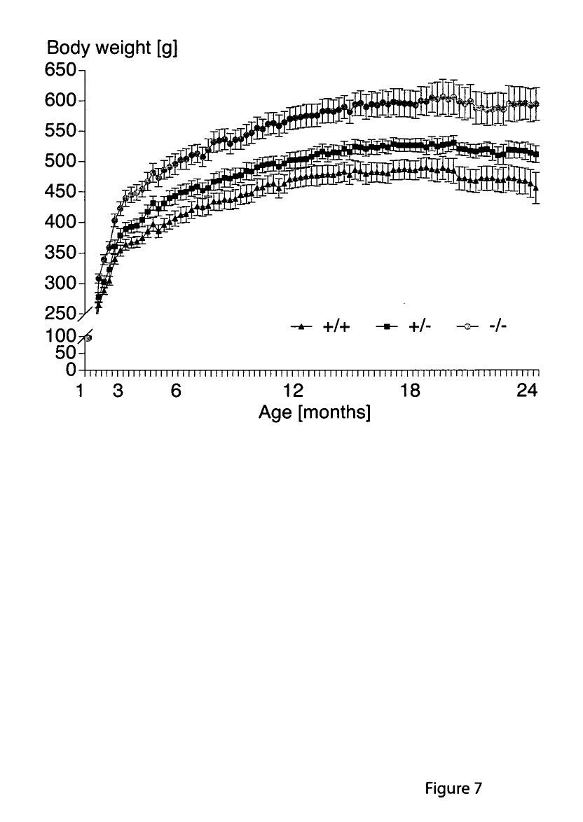

FIG. 7 shows the development of the body weight of wild-type control rats (−/−; n=10) in comparison to heterozygous HD (+/−, n=15) and homozygous (+/+, n=15) Huntington Disease (HD) transgenic (tg) rats over a period of two years (end of examination) with weekly points of measurement. Analysis of variance for repeated measurements reveals a significant effect of the factor “genotype” at F(3, 32): 12.4, p=0.0004), a significant effect of the factor for repeated measurement of body weight at F(2, 63): 882, p<0.0001, and a significant interaction of the factors (genotype=body weight) at F(2, 126): 3.6, p<0.0001. The significant interaction in ANOVA comes about by an increasing slowing down of body weight gain in HDtg rats over the period of measurement. At the age of 6 months, the HDtg rats are 5% lighter and at the age of 24 months weigh about 20% less than control animals. All data points represent mean values±standard error. This monitoring demonstrates the differential effect of the transgene on the growth rate and slow progress of the disease.

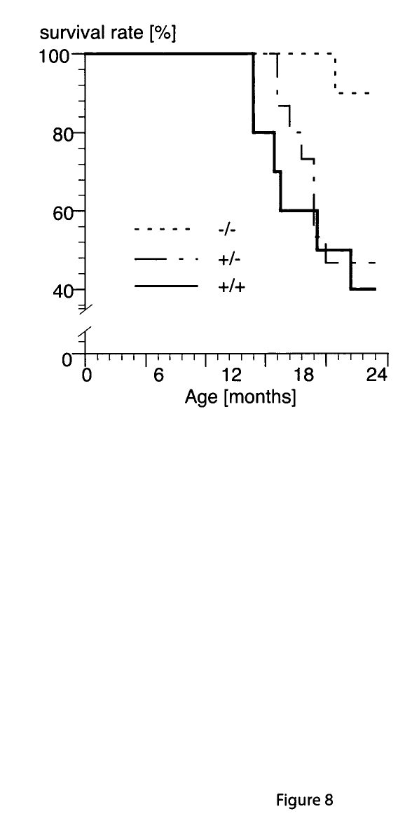

FIG. 8 illustrates the cumulative survival rate of male wild-type control rats (−/−; n=10) in comparison to heterozygous (+/−, n=15) and homozygous (+/+; n=15) Huntington disease (HD) transgenic (tg) rats over a period of two years (end of study) with monthly measurement points. The “log rank (mantelcox) test” for “event time in months” in the Kaplan-Meier analysis with chi square of 6.2 at a degree of freedom of two gives a significant difference of groups at p=0.04. This monitoring proves the effect of the transgene on the survival rate of the HDtg rats.

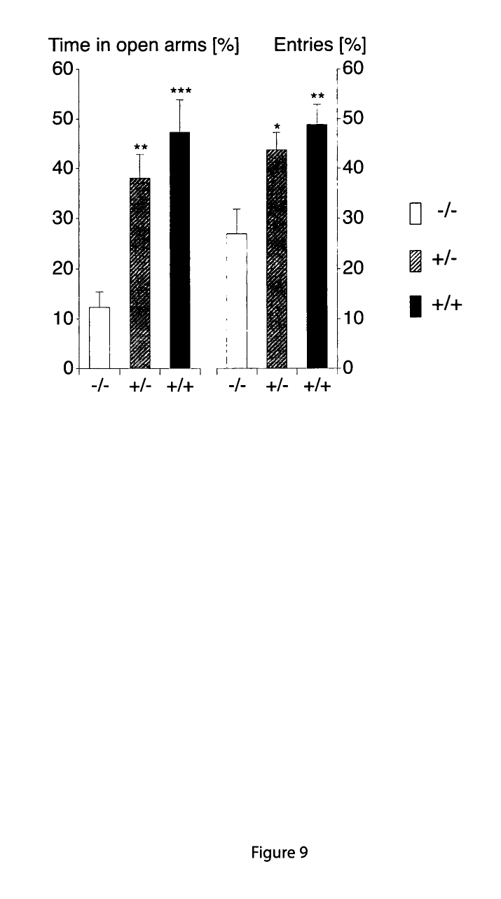

FIG. 9 shows the behavioral changes in “elevated plus maze test of anxiety” of male wild type control rats (−/−; n=10) in comparison to heterozygous (+/−; n=15) and homozygous (+/+; n=15) HDtg rats at the age of two months. The percentage of time in the open arms of the labyrinth is a well-validated parameter of anxiety in rodents. Increased and extended visits in the open arms prove an anxiolytic-like effect. HDtg rats (+/−, hatched columns and +/+, black columns) spend significantly more time (**p<0.001; ***p<0.0001) in the open arms. Also, the number of entrances into the open arms is increased (*p<0.01; **p<0.001). No difference was seen in the activity of the animals. This behavioral assay proves the differential effect of the transgene onto the emotional parameter “anxiety” in HDtg rats and therefore is comparable with findings in R6/2 mice and early emotional symptoms in HD patients.

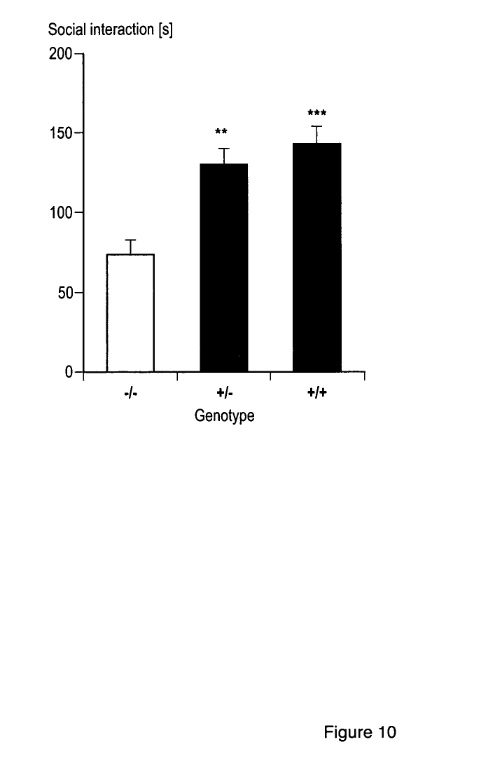

FIG. 10 shows behavioral changes in the “social interaction test of anxiety” of male wild type control rats (−/−; n=11) in comparison to heterozygous (+/−; n=13) and homozygous (+/+; n=11) HDtg rats at the age of ten months. The period of time which the animals spend in active social interaction in a novel environment with a partner test rat of the same genotype is an indicator for anxiety in rodents. Data represent mean values (±standard deviation) of the sum of time periods in active social interaction of both test animals. Prolonged active social interaction is regarded as an indicator for an anxiolytic-like effect. Analysis of variance revealed a significant effect of the factor “genotype” with F(2, 32): 12.4, p=0.0001. The HDtg rats (+/−, hatched columns and +/+, black columns) spend significantly (**p=0.0004; ***p<0.0001) more time in active social interaction. These studies demonstrate the effect of the transgene on the emotional parameter “anxiety” in a further behavioral assay, which in contrast to the “elevated plus maze test of anxiety” is also suitable for repeated examinations of progress control for the emotional parameters.

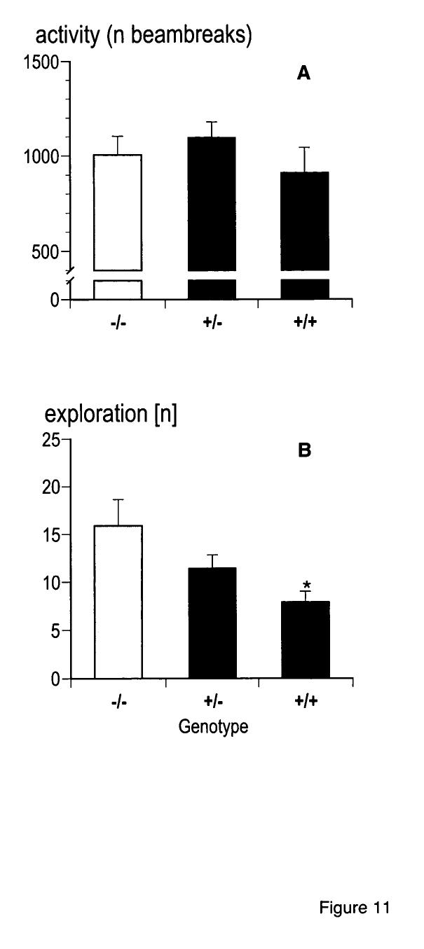

FIG. 11 shows the behavioral alterations in the “holeboard test of exploratory behavior” of male wild-type control rats (−/−; n=10) in comparison to heterozygous (+/−; n=15) and homozygous (+/+; n=10) HDtg rats at the age of three months. The number of movements of the head into the holes in the bottom of the testing apparatus (vertical activity) serves as a measure for exploratory behavior (curiosity) of the animals, while the number of interruptions of photoelectric barriers in the horizontal plane reflects general physical activity (A). Analysis of variance shows significant effects for the factor “genotype” with F(2, 32): 4.3, p=0.023. The homozygous HDtg rats (+/+, black columns) explore the holes in the bottom significantly less frequently (*p=0.006), with unchanged motor activity compared to wild type controls (B). This behavioral assay proves the differential effect of the transgene in HDtg rats on the emotional/cognitive parameter “exploration”. The results are comparable to early emotional symptoms in HD patients.

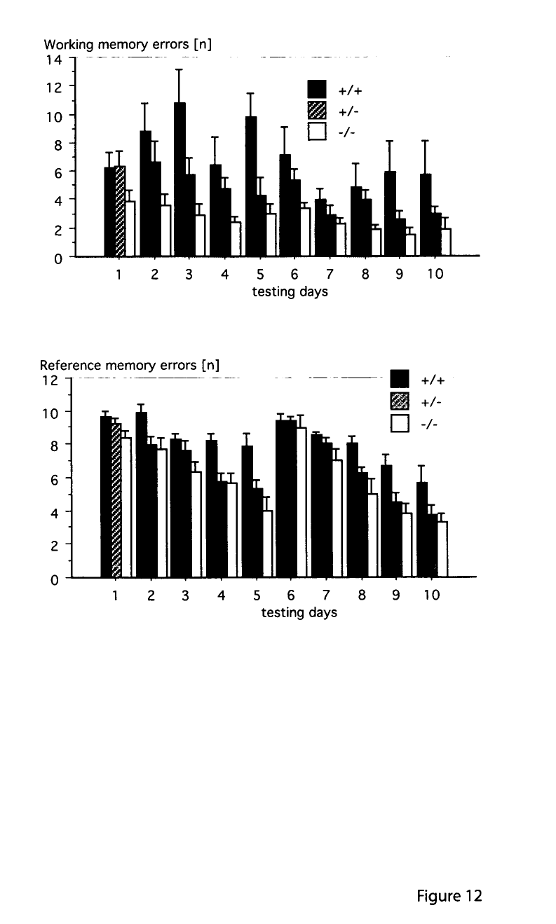

FIG. 12 shows the behavioral changes in the “radial maze test of spatial learning and memory” of male wild type control rats (−/−; n=9) in comparison to heterozygous (+/−; n=14) and homozygous (+/+; n=10) HDtg rats at the age of ten months. An “allocentric reversal” design was tested. Initially, there were four arms chosen randomly of the eight-arm radial maze rewarded with food pellets. These rewarded arms were not changed during the first five days. On testing day six, other arms were rewarded with food. The starting arms were randomly chosen from the non-rewarded arms. Orientation within the maze was “allocentric” for the rats, i.e. using visual cues outside the maze (walls, shelfs, doors, etc.). “Egocentric” information (e.g. a strategy like “always use every second arm to the right”) could not be used by the animals because the starting arms were chosen randomly. The animals were tested four times on one day. The number of multiple visits to arms visited before within one trial (working memory errors) (A) and the number of visits into unrewarded arms (reference memory errors) (B) were scored and mean values per testing day were calculated from four runs. Analysis of variance for repeated measurements revealed significant effects in both analyses for the factor “genotype” with F(3, 30): 7.7, p=0.002 for “working memory errors” (A) and with F(3, 30): 14.5, p<0.0001 for “reference memory errors”, as well as significant effects each for the factors of the repeated measurement proving the learning process. Post-hoc analyses showed significantly increased error numbers in the transgenic rats of both genotypes with p<0.001 each. All data points represent means±standard deviation. The results prove a “working memory amnesia” (impairment of the working memory), which then also hinders the acquisition of a “reference memory” (long term memory). This behavioral test proves the effect of the transgene in HDtg rats on the cognitive parameter “spatial learning” and is, therefore, comparable to findings in R6/2 mice and to the cognitive symptoms in HD patients.

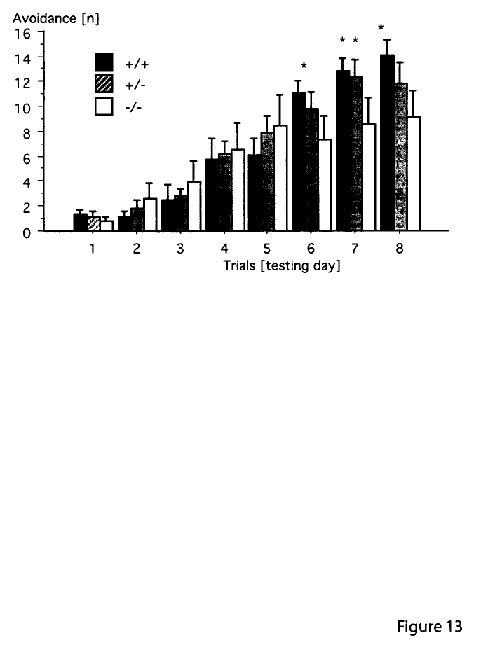

FIG. 13 shows the behavioral changes in the “two way active avoidance shuttle box test of associative learning” (associative learning in two-way active avoidance transfer chambers) of male wild type control rats (−/−; n=9) in comparison to heterozygous (+/−; n=13) and homozygous (+/+; n=10) HDtg rats at the age of nine months. Associative learning capabilities were tested within the framework of a coordination procedure. During multiple runs on consecutive days, the animals learned to avoid an announced (light or sound) aversive stimulus (electric shock) by active performance (transfer movement to a different compartment) by themselves. In contrast to the radial maze, the test is characterized by a high stress level. The number of correct avoidance reactions, the “active avoidance” (transfer into the “safe” compartment after the signal stimulus and before the aversive stimulus) is counted. Analysis of variance for repeated measurements show significant effect for the factor “avoidance” with F(3, 30): 7.7, p=0.002 and a significant interaction between genotype and avoidance with F(3, 30): 14.5, p<0.0001, proving a differential process of learning between the different genotypes. One factorial analysis of variance with consecutive post hoc analysis show a significantly increased number of avoidance responses in transgenic rats of both genotypes with p<0.01 each from the sixth day on. All data points represent mean± standard deviation. Results illustrate that under stress, the transgenic rats achieve a simple associative learning task better, which is often observed after functional impairments of the hippocampus. The results of these behavioral tests could prove pioneering for an enlarged insight into the pathology of HD, since they are indicative for an important role of stress regulating systems and prove a connection of the basal ganglia to the hippocampus.

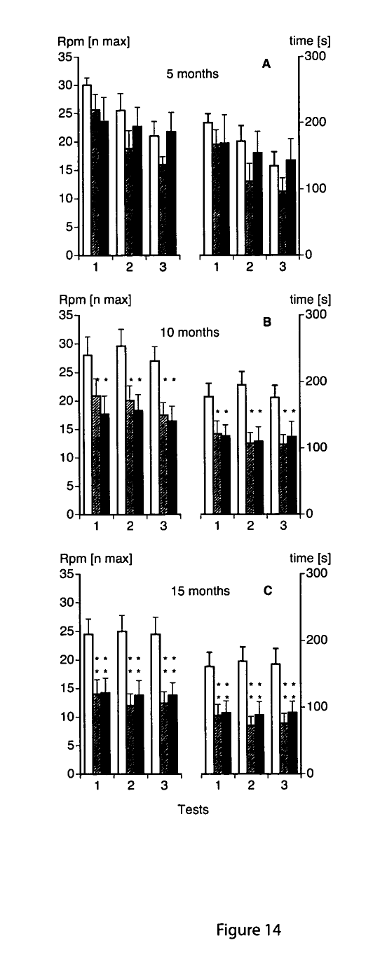

FIG. 14 shows behavioral changes in repeated “accellerod tests of motor coordination” of male wild type control rats (−/−; n=8) in comparison to heterozygous (+/−; n=10) and homozygous (+/+; n=9) Huntington's Disease (HD) transgenic (tg) rats. After a training period of five days, the ability to stay on an accelerating rotating rod, constantly rotating at 4 to 40 rotations per minute was evaluated in three trials per day on three consecutive days. The time periods in seconds until falling off and the maximally attained speed of the rod in rotations per minute (rpm [n max]) were averaged for each testing day and recorded for 5, 10 and 15 months old HDtg rats. Analysis of variance for repeated measurements of the tests at the age of 5 months revealed no significant effect of the factor “genotype” (A). At the age of 10 months (B), significant effects show for the factor “genotype” in time periods on the rotating rod with F(2, 24): 4.6, p=0.02 and for the maximally attained speed with F(2, 24): 4.2, p=0.03. At the age of 15 months (C), significant effects show for the factor “genotype” in the time period on the rotating rod with F(2, 24): 6.6, p=0.005 and the maximally attained rotating speed with F(2, 24): 7.0, p=0.004, proving a progressive decline in motor performance of the HDtg. This progressive deterioration of the parameter “balance and motor coordination” is found in a well validated behavioural test, which is suitable also for repeated examinations as a progress control. The motor impairments of HDtg rats come to a full markedness later in comparison to the point in time of appearance of emotional and/or cognitive alterations, which represents a further parallel to human HD.

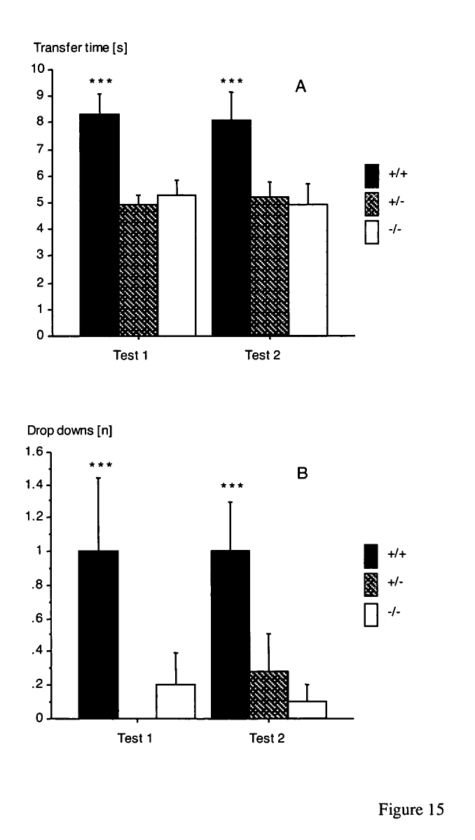

FIG. 15 shows behavioral changes in the “beam walk test of motor functions” of male wild type control rats (−/−; n=10) in comparison to heterozygous (+/−; n=14) and homozygous (+/+; n=10) HDtg rats at the age of five months. After a training period of three days on two further consecutive days the ability was tested to transfer on a thin beam and to hold on, if necessary. Different round and square (125 cm length) beams were used. The time for transfer in seconds and the frequency of drop downs were recorded. Analysis of variance for repeated measurements for the tests on a round beam of 16 mm diameter showed a significant effect of the factor “genotype” with F(2, 31): 8.1, p=0.0015 in the time period for transfer and for the frequency of drop downs with F(2, 31): 5.4, p=0.0096. Post hoc analyses by PLSD tests revealed that the statistical overall effect in the test of motorfunction was due to a significant (**=p<0.0001) deterioration in homozygous HDtg. These differential disturbances of motor function at the age of five months between hetero- and homozygous HDtg rats illustrate that the onset of and possibly also the specificity of impairments of motor function depends on the gene dose.

### EXAMPLES

### Example 1

First, a nucleic acid construct according to FIG. 1 was generated.

The construct controls via its regulatory unit where (topographically: in which tissue) and when (ontogenetically: embryonic or adult) the corresponding gene therebehind is switched on. In the present HDtg construct, the native rat huntingtin promoter was used as regulatory unit (FIG. 1B). The rat huntingtin promoter has been characterized and described in detail in the previous work of the inventors (Holzmann et al. 1998). As the second important component of the construct, the gene itself, a part of the rat huntingtin gene isolated by the inventors was used in the HDtg rats (Schmitt et al. 1995). Said rat huntingtin gene carries a disease-specific mutation generated from a patient's DNA by means of PCR. This was done by replacing the first 154 bp of a truncated huntingtin cDNA of the N-terminal rat sequence (RHD10) by the PCR product from the allele of an affected HD patient (FIG. 1B). The third component is the polyadenylation signal, which allows after transcription the addition of a poly(A) tail to the mRNA, thus providing the mRNA with stability against degradation processes (FIG. 1B). The whole construct is inserted into a vector in order to firstly replicate the construct in bacterial host cells and to generate as many copies of the total construct as possible. Said copies will then be injected into the male pronucleus of the fertilized ovum. The offspring derived from the re-implanted transgenic ova is further bred, and the expression and the function of the transgene in the animals are characterized (FIG. 2 and following).

Material and Methods for the Generation of the HDtg Rats (Example 1)

Generation of the Construct

To generate the construct, PCR was performed using DNA from a HD patient having 51 CAGs by means of the primer Hu 4 (ATGGCGACCCTGGAAAAGCTGATGAA) and Hu3-510 (GGGCGCCTGAGGCTGAGGCAGC). The resultant PCR product was subsequently digested with Eco811. The first 154 nucleotides of the cDNA RHD10 containing nt 1-1962 of the rat huntingtin gene (Schmitt et al., 1995) were removed by restriction of the clone with EcoRV and Eco81I. The resulting fragment was supplemented by the PCR product (FIG. 1B). Subsequently, a 885 bp rat huntingtin promoter fragment from position −900 to −15 (Holzmann et al., 1998) was ligated upstream of the cDNA, and a 200 bp fragment containing the SV 40 polyadenylation signal was added downstream of the cDNA, together resulting in the RHD/Prom51A construct (FIG. 1B). All cloning steps were controlled by sequencing. The construct replicated by the cloning vector was excised with XbaI and SspI out of the vector, microinjected into the male pronucleus of oocytes from Sprague-Dawley (SD) rat donors (Mullins et al., 1990; Schinke et al., 1999) and autologously intrauterinely reimplanted. After the rats had carried to term their litters, DNA was extracted from tail biopsies of each of the offspring according to standard procedures. Southern blot analysis of EcoRI digested DNA were performed to identify the transgenic “heterozygous” founder animals. This way, two transgenic animals as founders of the lines 2772 and 2762 were identified and subjected to further analysis, characterization and breeding steps (FIG. 2 and following).

Control of Huntingtin Expression

To control the expression of the transgene, Western blot analysis on brain samples were performed (FIG. 2). For Western blot analysis, frozen brain halves were homogenized, and protein was extracted under the protection of proteinase inhibitors using an ultra-turrax. After the addition of Nonidet P-40 (final concentration 1%), the homogenate was incubated for 15 minutes on ice. The protein extracts (30 μg/lane) were subjected to SDS-PAGE (4%) and blotted electrophoretically onto Immobilon-P membranes (Millipore). An already colored control marker for identifying different protein sizes (Novex Multimark, Invitrogen, Karlsruhe, Germany) was applied in a further lane. Wild-type and huntingtin protein were detected using the polyclonal anti-huntingtin antibody 675 (dilution 1:1,000) (Schmidt et al., 1998).

Western blot analysis on brain tissue of wild-type and HDtg rats of line 2771 (heterozygous animal) and line 2762 (heterozygous and homozygous animal): Polyclonal anti-huntingtin antibody 675 was used and revealed a reaction product having a size of 75 kD, confirming the expression of the transgene at a lower level than the endogenous protein. Homozygous rats express about double the amount of the transgenic protein compared to heterozygous animals.

Results (Example 1)

The rat HDcDNA fragment consisting of 1963 bp (Schmitt et al., 1995) carrying an expansion of 51 CAG repeats under the control of 885 bp of the endogenous rat HD promoter (Holzmann et al., 1998) (FIGS. 1A,B) could be successfully used for the microinjection. Two transgenic founder animals (founders) were carried to term after the re-implantation of the oocytes. The two founders were successfully used to establish the further breeding. Line 2762 was further characterized for more than 2 years. In this line, the CAG repeats remained stable in more than 147 meioses, and the Western blot (FIG. 2) provided evidence of the expression of the transgene.

Discussion (Example 1)

The results manifest the generation of HDtg rats and the expression of the transgenic huntingtin in the brain. This represents the first successful generation of a transgenic rat line for a human neurodegenerative disorder.

### Example 2

Introduction: Identification of Pathognomonic Changes in the Central Nervous System of HDtg Rats

In the preceding example, we have described the generation of HDtg rats and the expression of the transgenic huntingtin in the brain of two transgenic rat lines. In the following example 2, the identification of HD-specific changes in HDtg rats of line 2762 is described in detail. The description comprises (1) inclusion bodies and neurophilic aggregates in the striatum by immunohistology, (2) neurochemical alterations of tryptophane metabolism and its kynurenine, catechol and indoleamine metabolites in the CNS by HPLC analysis, (3) enlarged ventricles and focal lesions in the striatum by MRT scans, and (4) reduced glucose utilization in the striatum and in the cortex by PET studies.

Materials and Methods of Neuropathological Examinations and Neuroimaging (Example 2)

Immunohistological Identification of Inclusion Bodies and Neurophilic Aggregates

While under deep anesthesia, the brains of 18 months old HDtg rats and corresponding wild-type controls were perfused with buffer solution (PBS) at pH 7.2 for 30-60 seconds via the left ventricle of the heart. This procedure was followed by a perfusion with a 4% paraformaldehyde solution in 0.1 M phosphate buffer (PB) at pH 7.2 until completion of fixation. After removal, the brains were post-fixed for 6-8 hours in the same fixation solution and then stored in phosphate buffer with 0.1% sodium azide at +4° C. Free-floating sections of the brains were pre-treated with 4% normal goat serum in PBS with 0.1% Triton-X and avidin (10 μg/ml) to obtain a reduction of unspecific bindings of the antibody. Subsequently, the sections were incubated with the antibody EM48 (1:400 dilution) at 4° C. for 24 hr (Gutekunst et al., 1999; Li et al., 2000). Then, EM48 positive immunoprecipitations were transformed into complexes visible with a light-microscope using the ABC-method (avidin-biotin complex kit; vector ABC Elite, Burlingame, Calif., USA). An Axioscope 2 microscope with a digital camera was used for image processing. Wild-type rats of corresponding age served as controls.

Neurotransmitters in the Brain

Tryptophane and its kynurenine, catechol and indoleamine metabolites were measured by a newly developed and sensitive HPLC, as described previously (Vaarman et al., 2000). The brain was dissected into single brain regions and striatum and parietal cortex were stored at −80° C. Frozen brain samples were weighed and homogenized for 30 s in 100-500 μL 1.1 M perchloric acid. The homogenates were centrifuged at 13000×g at +4° C. for 20 min and 20 μl of supernatant was injected into a high pressure liquid chromatography (HPLC) system (ESA model 5600 CoulArray module with pump model 582 and autosampler model 540, Chelmsford, M A). Two coulometric array cell modules, each equipped with four working electrodes, were used. Detector potentials were as follows: channel 1-50 mV, 2-150 mV, 3-250 mV, 4-350 mV, 5-550 mV, 6-900 and 7-1000 mV. The pH value of the liquid phase was adjusted to 4.1, it was filtered and pumped at 0.5 mL/min. The chromatographic separation was achieved on an ESA MD-150 reversed-phase C₁₈ analytical column (particle size 3 μm, 150×3.0 i.d.) with a Hypersil pre-column (C₁₈, 7.5×4.6 mm i.d., 5 μm). All standards and reagents were of analytical purity grade and obtained via Sigma-Aldrich (St. Louis, Mo., USA) and Merck (Darmstadt, Germany), respectively.

Magnetic Resonance Tomography in HDtg Tats

The rats were anesthetized with 2% isoflurane and fixed in a stereotactic frame. Then, the animals were placed in the center of the MRT magnet. The magnetic resonance tomography was performed using a 4.7-T BRUKER Biospec tomograph. A whole body resonator allowed to obtain a homogeneous excitation field. The imaging procedure comprised 11 axial and 7 coronal images in cross-section over the whole brain having a thickness of 1.3-1.5 mm, a field of view of 3.2×3.2 cm and a matrix of 256×256 at TRITE 3000/19 ms for 6 average values. The images were analyzed by means of Scion Image Software (Scion Corporation, Maryland, USA).

Positron Emission Tomography (PET) in HDtg Tats

Nineteen adult male HDtg rats with wild type controls at the age of 24 months were examined. Among them were homozygous animals (+/+; n=6; 410-520 g; 451.7 g±17.6), heterozygous rats (+/−; n=7; 460-580 g; 502.1 g±16.1) and age-matched wild type controls (−/−; n=6; 537-660 g; 589.5 g+20.7). All animals featured identical standard breeding conditions. Immediately after completion of the PET studies, the [¹⁸F]FDG-PET studies were calibrated by ex vivo [¹⁸F]FDG measurements to verify the reliability of the in vivo measurements.

The PET studies were performed using a high-resolution small animal PET scanner (“TierPET”) (Weber et al., 2000). Precise identification of anatomical structures was guaranteed by parallel images of MRT images. For the high-resolution TierPET, the anesthesized animals received an injection of 0.3 ml [¹⁸F]FDG (1 mCi/ml, dissolved in NaCl 0.9%). In parallel, blood glucose measurements were performed. During the studies, the animals were placed onto a coordinate table for precise localization within the x-, y- and z-axis. For the ex vivo FDG autoradiography, the brains were removed, and coronal sections (20 μm) were made on a cryotom (CM 3050, Leica, Germany). Said coronal sections were exposed for 18 hours to a phosphor imaging plate (BAS-SR 2025, Fuji, Germany), together with calibrated [¹⁸F] brain tissue standard. Then, the imaging plates were scanned using a high-performance imaging plate reader (BAS5000 BioImageAnalyzer, Fuji, Germany) at a resolution of 50 μm and analyzed by means of specific software (AIDA 2.31, Raytest, Germany), taking into account standards, dosing, body weight, blood glucose levels, etc. The local glucose utilization rate (1CMR_{Glc}) (Sokoloff et al., 1977) was calculated according to Ackermann and colleagues (Ackermann et al., 1989) on the basis of the following constants: k₁=0.30; k₂=0.40; k₃=0.068; LC=0.60. The results of the FDG-PET study and the ex vivo autoradiography were analyzed by means of linear regression analysis and statistically evaluated.

Results of the Neuropathological Studies and Neuroimaging (Example 2)

Specific Accumulation of Huntingtin Aggregates and Nuclear Inclusion Bodies in the Striatum

To examine whether mutant huntingtin forms aggregates in the brain of 18-month-old HDtg rats, the antibody EM48 was used (Gutekunst et al., 1999; Li et al., 2000), which identifies specifically mutant huntingtin and its aggregates. FIG. 3 shows neuropathological alterations in frontal histological sections through the striatum of HDtg rats in form of nuclear inclusion bodies and neurophil aggregates. The most cases of EM48 positive immunoreactivity were found in the striatum, where it appeared as a huntingtin aggregate specific, punctuate staining in the striatum. In wild type controls, huntingtin aggregates were completely absent (FIG. 3A). The latter were in particular concentrated in the front region of the striatum (Str), in immediate proximity of the lateral ventricles (arrow) of HDtg rats (FIG. 3B).

Also in the nucleus caudatus of the striatum of the HDtg rats, many nuclear aggregates and small neurophil aggregates can be found (FIG. 3C). Neurophil aggregates (arrows) can also be observed in the lateral gyrus pallidus (LGP). Greater enlargements from the striatum of HDtg rats show EM48 positive nuclear aggregates (arrow heads) as well as small neurophil aggregates (FIG. 3D; arrows). Only few EM48 aggregates can be observed in the cortex (Ctx). Other regions of the brain such as the hippocampus and the cerebellum showed very weak or no immunoreactivity.

Two different types of EM48 staining can be observed: nuclear inclusion bodies and neurophil aggregates. Some neurophil aggregates are arranged in a pearl necklace-like fashion (FIG. 3C). This staining pattern is approximately identical to other animal models of the HD (Gutekunst et al., 1999). Single nuclear inclusion bodies are often observed in the striatum (FIG. 3D), and they can also be found in similar manner in other HD mouse models (Davie et al., 1997; Li et al., 2000). Since the axons of the striatal projection fibers terminate in the lateral globus pallidus (LGP), also the caudal region of the striatum was examined. Nuclear staining as well as neurophil aggregates are common in the striatum. In the LGP, however, mainly neurophil aggregates can be observed.

Postmortem Concentrations of Tryptophane and Biogenous Amines

To identify neurochemical alterations in the brain of HDtg rats, a new and sensitive HPLC method was used which allows for simultaneous determination of different transmitters from individual samples. At the age of 18 months, the striatal dopamine levels in heterozygous HDtg rats were decreased by 20%, whereas in the homozygous HDtg rats, they were decreased threefold (FIG. 4A). The levels of dopamine in the parietal cortex were not significantly different from the wild type controls (FIG. 4B). Likewise, the DOPAC levels were not changed (FIGS. 4D, E). In accordance with preceding notes with regard to changes of the tryptophane metabolism in HD, also in the HDtg rats a twofold decrease in the striatal tryptophane could be observed (FIG. 4E). Further, tryptophane tended to decrease in the parietal cortex (FIG. 4F). Interestingly, the levels of xanthurenic acid, a metabolite of 3-hydroxykynurenine having neuroprotective characteristics, were dramatically reduced in the striatum of homozygous HDtg rats (FIG. 4G) and undetectable in the parietal cortex (FIG. 4H). In contrast, the levels of xanthurenic acid were elevated in the parietal cortex (FIG. 4H), but unchanged in the striatum (FIG. 4G). All in all, these studies demonstrate that the neurochemical alterations in the CNS of the HDtg rats are comparable to those in the human HD.

Focal Lesions in the Striatum and Enlargement of the Lateral Ventricles in the Brain

To examine whether the HDtg rats also present neuropathological alterations detectable by neuroradiology, the above mentioned MRT studies were performed on 8-month-old female HDtg rats of different genotypes and wild type controls. Compared to the controls (FIGS. 5A, B), the homozygous transgenic female animals presented apparently enlarged lateral ventricles on coronal (frontal) (FIG. 5C, arrows) and sagittal (FIG. 5D, arrows) sections. The comparison of MRT scans on coronal level of the striatum of a wild type animal (FIG. 5E) and a HDtg animal (FIG. 5F) reveals apparent focal lesions in the striatum of the HDtg rat (arrows in FIG. 5F). Using the neuroradiological method MRT, these studies demonstrate that the HDtg rats present similar alterations compared to the human HD.

Quantitative in vivo Determination of the Local Brain Metabolism using High-Resolution Positron Emission Tomography (PET)

[¹⁸F]FDG and PET are used to determine the local metabolization of glucose 1CMR_{Glc}) in HD patients. Up to now, these studies have consistently revealed reductions in metabolization rates. In order to examine whether such alterations can also be found in the HDtg rats, and whether this animal model is therefore suited for a continuous in vivo monitoring, a study was performed using [¹⁸F]FDG and high-resolution small animal PET. The results were compared to MRT images to identify the regions of interest (ROI), and to ex vivo [¹⁸F]FDG measurements made immediately after the PET study.

Harderian glands, the olfactory bulb and different regions of the brain, such as the striatum and the caudato-putamen complex, respectively, are clearly distinguishable (FIG. 6). The figure is composed of representative images from the [¹⁸F]FDG small-animal PET in horizontal (B-D) and coronal (F—H) sectional planes along with individual MRT scans (A, E) and ex vivo autoradiographies (J, K). MRT scans (A, E) of a wild type control animal are co-registered with the respective [¹⁸F]FDG-PET images (B, F). MRT scans (A, E) of a wild type control animal are co-registered with the respective [¹⁸F]FDG-PET images (B, F). The planes are on the level of the caudato-putamen complex. The sectional planes for the autoradiography (J, K) are taken from the same animals as the [¹⁸F]FDG-PET images (B, F, D, H). The measurement ranges (regions of interest; ROI) within the [¹⁸F]FDG-PET images are defined using the corresponding MRT scans (as indicated by the white lines; FIGS. 6A, E). The local rate of glucose metabolism (1CMR_{Glu}) is absolutely quantified (see black-and-white scales; FIG. 6). The high accumulation of activity in the caudato-putamen area is clearly visible in [¹⁸F]FDG-PET images (F, G, H) and in the autoradiographies (J, K). The mean 1CMR_{glc} values were 54.98±15.53 [μmol/100 g·min] for the whole brain and reduced in heterozygous and homozygous transgenic rats (see legend of FIG. 6). Homozygous transgenic rats present significantly (p<0.05) reduced 1CMR_{Glu} values compared to the wild type controls, both in [¹⁸F]FDG-PET (34.54±18.52 vs. 54.98±15.53) and in the ex vivo autoradiographies (43.54±6.77 vs. 63.02±8.24). These studies demonstrate by means of the neuroradiological method PET that the HDtg rats present disorders in the utilization of glucose which are similar to the human HD. Further, the approach gives evidence that it is possible, by means of repeated PET analysis, to perform progression studies in HDtg rats which were not possible in mice up to now.

Discussion of the Neuropathological Studies and Neuroimaging (Example 2)

Although mice are still the species of choice for genetic manipulations, there are a number of questions which are more adequately answered in the rat. Among such appropriate methods are neuroradiological studies such as MRT and PET which can only be performed in rats or in bigger species due to the species size and allow for repeated determinations and consequently for progression studies.

Similar to HD mouse models, the present transgenic rat model of the HDtg rat reveals on the neuropathological level nuclear inclusion bodies and neurophil aggregates, in particular in the striatum (Wheeler et al., 2000; Li et al., 2000). On a neurochemical level, reduced tryptophane levels can be found, quite similar to those in HD patients (Stone, 2001). Further, particularly a nearly complete depletion of xanthurenic acid can be seen in the striatum and in the cortex of homozygous HDtg rats. In the less clearly afflicted heterozygous HDtg rats, however, there are still levels of xanthurenic acid. These findings give reason to assume the loss of an important neuroprotective mediator in the homozygous HDtg rats (Stone, 2001) and possibly give evidence for a protective compensation mechanism in the heterozygous HDtg rats against excitotoxic metabolites from an overactive indoleamine (2,3)-dioxygenase metabolism (Widner et al., 1999). Normal DOPAC levels and reduced levels of xanthurenic acid might be associated causally with an increased formation of quinolinic acid which presents neuroexcitant and neurotoxic properties. These findings support the hypothesis that both increased production of quinolinic acid (Bruyn et al., 1990) and decreased production of neuroprotective metabolites from tryptophane (Stone, 2001) may be relevant to the pathogenesis of HD.

The essential advantage of the present invention, however, is its suitability for in vivo neuroradiological methods which are not applicable with mice. The MRT images demonstrate, similar to the human adult form of the HD, an enlargement of the lateral ventricles due to shrinkages of the striatum. In addition, local lesions can be found there in the striatum, which could be interpreted as gliosis. Further, the glucose metabolism is significantly reduced. In late stages of the human HD, clinical studies consistently reveal a reduced 1CMRG_{Glc} in the striatum (Kuwert et al., 1990; Young et al., 1986). Thus, this example provides evidence that the present invention is a transgenic rat model which closely reflects the human neuropathology, and which is the only one so far to be suited for in vivo monitoring of neuroradiopathology, brain metabolism and other in vivo parameters, such as measurements of receptor density and enzyme activity. Finally it should be noted once more that the present animal model does not show—in contrast to the R6/2 mouse—any signs of diabetes mellitus.

### Example 3

Introduction: Characterization of Typical Behavioral Alterations in HDtg Rats

In the preceding examples, the generation of HDtg rats and the expression of the transgenic huntingtin in the brain of two transgenic rat lines (Example 1) as well as the identification of HD-specific pathognomic changes in the brain of line 2762 were described. In addition, the suitability of the present invention and the model, respectively, for neuroradiological methods such as MRT and PET could be proven. In the following example 3, the further characterization of the phenotype as well as the characterization of the behavior of the HDtg rats of line 2762 is described in detail. This comprises (1) monitoring of growth, reflexes, and lethality, (2) description of emotional alterations, (3) description of cognitive differences, and (4) description of deficiencies in motor function. In general, the characterization of the HDtg rats followed the principles for the characterization of mice with unknown phenotype (Crawley et al., 1998) with special adaptation to the specific needs for testing rats.

Method of Behavioral Phenotyping (Example 3)

Development, Evolution of Body Weight and Lethality

The animals were regularly examined with regard to their general health condition. Such examinations also comprised all tests for neurological reflexes and sensory perceptions mentioned in Crawley (1998). Monitoring the evolution of the body weight of wild type control rats (−/−; n=10) belonging to the Sprague Dawley strain by comparison with heterozygous (+/−; n=15) and homozygous (+/+; n=15) Huntington's Disease (HD) transgenic (tg) rats over a period of two years (end of the study) was reached by weighing the animals each week. The age of death was recorded and subsequently analyzed by means of the Kaplan-Meier statistics.

Elevated Plus Maze Test to Measure the Anxiety in Rodents

The elevated plus maze (EPM) is one of the most common paradigms to measure anxiety-induced behavior. It is of advantage that the test is an easy procedure which is simple to perform and which presents a high retest reliability for two test runs (Pellow et al., 1985) The EPM consists of two open arms and two further arms which are enclosed by side walls, disposed in the form of a “+”. It could be demonstrated that rats tend to stay in the closed arms. Both the number of entries and the duration of stay in the closed arms is higher than in the open arms. Animals which are placed into the EPM (typically at the crossing point of the four arms) rather enter one of the closed arms. This natural tendency can be reduced by anxiolytics (e.g. diazepam), leading to an increased number of entries and duration of stay in the open arms.

The EPM consists of a total of four arms (50×10 cm), with two opposite arms being enclosed by side walls (40 cm in height). A computer-based device equipped with light barriers was used, manufactured by TSE-Systems (Bad Homburg, Germany). The arms are 50 cm above the ground. The illuminance was 0.3 lux under red light conditions, and the tests were performed in the dark cycle of the animals. The behavior of the animals was recorded for 5 min. After each test run, the EPM is cleaned with 70% alcohol. Test parameters are: number of entries, duration of stay in the arms and in the center. Details of the test have already been described in comprehensive manner (Breivik et al., 2001). The percentage share of duration of stay and number of entries in the open arms is a well validated parameter for anxiety in rodents. Age-matched male wild type control rats (−/−; n=10) belonging to the Sprague Dawley strain were tested with heterozygous (+/−; n=15) and homozygous (+/+; n=15) HDtg rats at the age of two months. The increase in the number and duration of visits on the open arms provides evidence for an anxiolytic-like effect.

Active Social Interaction in New Environments (Social Interaction Test of Anxiety)

The social interaction test according to File (1998) is a test of anxiety which is not based on any deprivation model or highly aversive stimuli. Negative intensifications, such as electric shocks, are not used. To make the animals feel uncomfortable, they are placed in a new environment (and the light conditions are manipulated). The time that the rats spend in active social interaction reaches its maximum when the rats are placed in a well known environment with only low illumination. The decrease in SI time, however, is correlated with an increase in other behaviors indicating stronger emotionality, such as defecation and “freezing”. Thus, social interaction time is correlated with emotionality, and not with exploration.

The test arena was an open field of 50×50 cm that was placed in a sound isolation box. A white light bulb (60 watt) was used for illumination. The illumination level in the open field is between 175 and 190 lux. The behavior of the animals is recorded online using a video camera placed within the isolation box above the open field. The fields in which the animals enter and the SI time are recorded online. Subsequently, the frequencies of the individual behaviors are analyzed by means of the videotapings The animals are placed in the middle of the open field one after the other, and 10 sec later, data recording begins. The following parameters are recorded: duration of time spent in sniffing, following, crawling under and over other rats, but not passive body contact between the animals (such as resting and sleeping) without active social interaction. The method has been recently described in detail and validated in our laboratory (Kask et al., 2001). Male wild type control rats (−/−; n=11) were compared with heterozygous (+/−; n=13) and homozygous (+/+; n=11) HDtg rats at the age of ten months. The duration of time which the animals spend in active social interaction in a new environment with a partner test rat of the same genotype was interpreted as an indicator for anxiety in rodents. The statistical analysis by means of one-factorial analysis of variance (ANOVA; factor: genotype) is based on the totals of times in active social interaction of both test animals. Prolonged active social interaction is considered as an indicator for an anxiolytic-like effect.

Holeboard Test of Exploration

The holeboard test examines both the “directed” exploration and movement-dependent parameters of behavior (File and Wardill, 1975). Dipping the head into a hole in the ground is a spontaneously produced behavior of the rat, whose frequency represents the extent of curiosity (“inquisitive exploration”; Robbins und Iversen, 1973). The holeboard apparatus consists of wooden boxes (65×65×40 cm) with 16 holes in the bottom, equally spaced from each other. Each hole has a diameter of 3 cm. Light barriers are provided below said holes, communicating with a computer. Any head dip into a hole is automatically recorded. On horizontal level, a total of 25 squares of 13×13 cm is surrounded by light barriers located in the walls to record also any directed activity on this level. In the present studies, rats were transported to a soundproof testing room. The number of head dips was recorded and interpreted as directed activity in the perpendicular vertical. Also the locomotive activity was determined. Male wild type control rats (−/−; n=10) were compared with age-matched heterozygous (+/−; n=15) and homozygous (+/+; n=10) HDtg rats at the age of three months. The number of head dips into the holes in the bottom of the test apparatus (vertical activity) is used to evaluate the exploration behavior (curiosity) of the animals, whereas the number of interruptions of the light barriers on horizontal level reflects the general physical activity. The test was conducted without prior habituation during the dark cycle. The method has recently been described in detail (Breivik et al., 2001). The test data were recorded in Excel tables before transmitting them to a statistical program (Stat View 5.0) and analyzing them by means of ANOVA for repeated measurements with the factor “genotype” on a Macintosh G3 computer. This data preparation was followed by an analysis by means of one-factorial ANOVA with Fischer's PLSD post-hoc test, if useful.

Spatial Learning in the 8-Arm Maze (Radial Maze)

In the radial 8-arm maze, the test animal, previously deprived of food, was rewarded with food in predetermined arms of the maze. The test for the animal is to remember the arms which were rewarded during the previous run as well as the arms the animal already explored during the actual run. For this purpose, the rat has to learn the spatial relations between the arms of the maze as well as the cues from the spatial environment. The radial maze manufactured by the company TSE Systems consists of an octagonal base plate carrying the eight arms which are mounted in a star-like manner (550/425×150/145×225 mm; L×W×H). The walls of the arms are made of non-transparent grey plastic. On the front walls of the arms, a food pellet is provided in a cup to receive the food. A sensor is disposed in the cup for the detection of food removal. Each arm is provided with a special light barrier array, arranged approx. 10 cm behind the entrance of the arm (three near the ground and the forth light barrier in the center, above the three others). At these places, the side walls of the arms are interrupted so as to determine by means of the light barriers if the animal is in an arm or in the center. Data recording is made using a control unit manufactured by TSE Systems GmbH, Bad Homburg, Germany, which transmits the data directly to a computer equipped with appropriate software. Age-matched male wild type control rats (−/−; n=9) were compared with heterozygous (+/−; n=14) and homozygous (+/+; n=10) HDtg rats at the age of ten moths. An “allocentric reversal” design according to Hölscher and Schmidt (1998) was tested. After habituation and exploration tests (data not shown), four randomly chosen arms of the eight-arm radial maze were rewarded with food pellets. These baited arms were not changed for the first five days. On the sixth test day, other arms were rewarded with food. The starting arms were randomly chosen from the non- rewarded arms. Orientation in the maze was “allocentric” for the rats, that is by visible cues outside the maze (walls, shelves, door, etc.). The starting arms being randomly chosen, “egocentric” information (such as a strategy like “always turn right at each second arm”) was not applicable for the animals. The animals were tested four times the day. The number of repeated visits to previously visited arms during the same test (working memory errors) and the number of visits to non- rewarded arms (reference memory errors) were recorded, and mean values per test day were calculated from four runs. An analysis of variance for repeated measurements via the factor “genotype” and for the repeated measurement of the respective parameter was performed. If significant interactions between the factors were observed, said analysis of variance was followed by a one-factorial variance analysis for each individual test day via the factor “genotype”. In the case of significant overall effect interactions, it was further followed by a posthoc-analysis (Fisher's PLSD).

Associative Learning in “Two-Way Active Avoidance Transfer Chambers”

(Two Way Active Avoidance Shuttle Box Test of Associative Learning)

Shuttle box learning with active avoidance of an aversive stimulus is a typical, well established and validated test for associative learning ability in rats. A TSE shuttle box system (Technical & Scientific Equipment GmbH, Bad Homburg, Germany) was used, which allowed active and passive avoidance learning experiments in rats. It consists of two compartments communicating with each other via a door and provided with light barriers, a control unit and computer equipped with control and acquisition software. An electric shock, the unconditioned stimulus, is applied via a metal grid on the bottom of the boxes. The conditioned stimulus may be a single stimulus (sound or light) or a paired stimulus (sound with light). The tests were performed in male wild type control rats (−/−; n=9), heterozygous (+/−; n=13) and homozygous (+/+; n=10) HDtg rats at the age of 9 months. Associative learning ability in the frame of a conditioning process was tested. In multiple runs on successive days, the animals learn to avoid a signaled (light or sound) aversive stimulus (electrical shock) by their own activity (locomotion to another compartment). In contrast to the radial maze, this test is characterized by a high stress level. The number of correct avoidance reactions, i.e. the “active avoidance” (locomotion to the “safe” compartment after the signal stimulus and before the aversive stimulus) is counted. The data were analyzed using the analysis of variance for repeated measurements via the factor of the repeated measurement “avoidance” and the factor “genotype”. This analysis was followed by one-factorial analysis of variance with subsequent posthoc analysis for each individual test day. All data points represent mean values±standard errors.

Motor Performance Test on an Accelerating Rotating Rod (Accelerod Test)

The rotarod test and its sub-form, the accelerod test, are the most frequently used tests to verify the neuromotorical abilities and the balance of rodents. The rotating rod apparatus used for the present studies is manufactured by TSE Systems (Bad Homburg, Germany). The rod has a diameter of 7 cm and a total length of 50 cm and is divided by five disks into four sections. Each section has a width of 12.5 cm (applicable for rats). This allows to test simultaneously four rats per run. To prevent the rats from jumping off the rod, the latter is located 26 cm above the ground. The larger the distance between the rod and the ground, the higher the motivation of the animals to stay on the rod. Using the device in the accelerod mode means that the speed and the number of rotations of the rod will automatically and continuously increase to a specific adjustable maximum value (e.g. from 4 rpm/min in steps of four rotations to a final of 40 rpm/min in 5 min). If the rotational speed is maintained constant and then gradually increased, the rotarod mode is used. The motor performance test of the animals is divided into a training phase and two test phases. Test parameters are latency until falling off the rod in seconds and maximal reached rotational speed in rotations per minute (rpm). During the training phase, the animals are placed on five days, two times per day, for respectively two minutes onto the rotarod at a rotational speed of 20 rpm. If an animal falls off the rod during the training phase, it is replaced onto the device after 10 sec. There are two runs with an interval of one hour. The test animals are placed onto the rod not more than five times per run. The rollers are cleaned after each training run. During the accelerod test phase, the animals are placed onto the apparatus at a low rotational speed (40 rpm). The apparatus is set to accelerod mode and accelerates within 4.5 min to the highest rotational speed (40 rpm), with each run having a duration of maximal 5 minutes (then, the animals are removed from the rod). It is recorded when and at which speed the animals fall off the rod. The tests are performed on three successive days with three runs each at an interval of two hours. Male wild type control rats (−/−; n=8) were compared with heterozygous (+/−; n=10) and homozygous (+/+; n=9) Huntington's disease (HD) transgenic (tg) rats at the age of five, ten and fifteen months. Mean values are determined from the results of each test day. Then, the results over three successive test days are subjected to an analysis of variance for repeated measurements. Subsequently, the differences between the individual groups are determined by means of the one-factorial ANOVA with posthoc test, if necessary.

Motor Performance Test in the Beam Walk Test

During this frequently used test for the motor performance, rodents have to traverse a beam of different diameter, characteristics and length to get from one compartment into another. In the present test, the beam consisted of a circular non-coated rod made of wood with a diameter of 16 mm and a length of 125 mm, which was horizontally placed 60 cm above the ground and bridged two compartments. The starting compartment was a white, well lightened box, and the destination compartment was a black, darkened box. After a training phase of three days, the ability to traverse this narrow beam and, if necessary, to hold tight on it was tested during two further successive days. Age-matched male wild type control rats (−/−; n=10), heterozygous (+/−; n=14) and homozygous (+/+; n=10) HDtg rats at the age of five months were compared. The time required to traverse the beam in seconds and the frequency of falling off was recorded. This was followed by an analysis of variance for repeated measurements.

Results of Behavioral Phenotyping (Example 3)

Development, Evolution of the Body Weight and Lethality

At birth, HDtg rats are indistinguishable from their wild type littermates. Offspring of both sexes are fertile, and no evidence for atrophy of the sexual organs was found. At all measurement times, the blood glucose levels were within the physiological age-dependent standard range. Throughout the first three months of life, the transgenic animals are about 5% lighter than their wild type littermates. In some cases, HDtg rats show opisthotonus-like movements of the head, and in six of 280 rats examined up to now, circling behavior could be observed which disappeared at the age of about one year. At no time, resting tremor, ataxia, knocking together of the legs (clasping), abnormal uttering, dyskinesia or seizures were observed.

The evolution of the body weight of wild type control rats compared to heterozygous and homozygous HDtg rats is illustrated in FIG. 7. The analysis of variance for repeated measurements reveals a significant effect of the factor “genotype” with F(3, 32): 12.4, p=0.0004, and a significant effect of the factor for repeated measurement of body weight F(2, 63): 882, p<0.0001 as well as a significant interaction of the factors (genotype x body weight) with F(2, 126): 3.6, p<0.0001. The significant interaction in the ANOVA is achieved by the more and more slower body weight gain in the HDtg rats throughout the measuring period. At the age of six months, the HDtg rats are 5% lighter, and at the age of 24 months, they are 20% lighter than the control animals. FIG. 8 shows the cumulative survival rate of wild type control rats (−/−; n=10) belonging to the Sprague Dawley strain, compared with heterozygous (+/−; n=15) and homozygous (+/+; n=15) Huntington's disease (HD) transgenic (tg) rats over a period of two years (end of study) with monthly recording times. The “Log-Rank (MantelCox) Test” for “Event time in months” from the Kaplan-Meier analysis reveals with a Chi square of 6.2 at a degree of freedom of 2 a significant group difference with p=0.04. The reason for cachexia and death is not clear up to now. However, the number of tumors in transgenic rats at different locations is increasing.

Elevated Plus Maze (EPM)

The behavioral alterations in the EPM are illustrated in FIG. 9. HDtg rats (+/−, hatched columns and +/+, black columns) spend significantly (**p<0.001; ***p<0.0001) more time on the open arms. Also the number of entries into the open arms (*p<0.01; **p<0.001) is increased. No differences in the activity of the animals were found.

Social Interaction Test of Anxiety

FIG. 10 shows the behavioral changes in the “Social interaction test of anxiety”. The data represent mean values (±standard errors) derived from the totals of the time spent in active social interaction of both test animals. Prolonged time spent in active social contact is considered as an indicator for an anxiolytic-like effect. The analysis of variance reveals significant effects for the factor “genotype” with F(2, 32): 12.4, p=0.0001. The HDtg rats (+/−, hatched columns and +/+, black columns) spend significantly more time (**p=0.0004; ***p<0.0001) in active social interaction.

Holeboard Test of Exploration

FIG. 11 shows the behavioral changes in the “Holeboard test of exploratory behavior”. The analysis of variance reveals significant effects for exploration and curiosity, respectively, for the factor “genotype” with F(2, 32): 4.3, p=0.023. The homozygous HDtg rats (+/+, black columns) explore the holes in the bottom of the test apparatus (FIG. 11A) significantly less frequently (*p=0.006) while motor activity remains unchanged in comparison to the wild type controls (FIG. 11B).

Spatial Learning in the 8-Arm Maze

The results for spatial learning in the “radial maze test of spatial learning and memory” are illustrated in FIG. 12. The analysis of variance for repeated measurements reveals significant effects in both analysis for the factor “genotype” with F(3, 30): 7.7, p=0.002 for “Working memory errors” (FIG. 12A) and with F(3, 30): 14.5, p<0.0001 for “Reference memory errors” (FIG. 12B) as well as significant effects for the factors of the repeated measurement, proving the learning process. Posthoc analysis reveal a significant increase in the number of errors in the transgenic rats of both genotypes with p<0.001 each. All data points represent mean values±standard errors.

Associative Learning in the “Two-Way-Active-Avoidance-Transfer-Compartments”

The results of the shuttle box tests for associative learning are summarized in FIG. 13. During multiple runs on successive days, the animals learn to avoid a signaled (light or sound) aversive stimulus (electric shock) by their own activity (locomotion to another compartment). The number of correct avoidance reactions, the “active avoidance” (locomotion to the “safe” compartment after the signal stimulus and before the aversive stimulus) is illustrated. The analysis of variance for repeated measurements reveals significant effects for the factor of the repeated measurement “avoidance” with F(3, 30): 7.7, p=0.002 and a significant interaction between genotype and avoidance with F(3, 30): 14.5, p<0.0001, indicating a differential learning process between the individual genotypes throughout the repeated tests. One-factorial analysis of variance with subsequent posthoc analysis show significantly increased avoidance reactions in the transgenic rats of both genotypes with p<0.01 each as of the sixth day of testing.

Motor Performance Test in the Accelerod Test

The results of the repeated accelerod tests are presented in FIG. 14. After a training period of five days, the ability to stay on a rotating rod with a constant acceleration from 4 to 40 rotations per minute was measured during three tests per day on three successive days. The time in seconds until falling off and the maximal achieved rotational speed of the rotating rod in “rotations per minute” (rpm [n max]) were measured in 5-, 10- and 15-month old HDtg rats. The analysis of variance for repeated measurements shows for the tests in 5-month-old HDtg rats no significant effect of the factor “genotype” (FIG. 14A). At the age of 10 months (FIG. 14B), significant effects for the factor “genotype” for the time spent on the rotating rod with F(2, 24): 4.6, p=0.02 and the maximal achieved rotational speed F(2, 24): 4.2, p=0.03 are found. In 15-month-old animals (FIG. 14C), significant effects for the factor “genotype” for the time spent on the rotating rod with F(2, 24): 6.6, p=0.005 and the maximal achieved rotational speed with F(2, 24): 7.0, p=0.004 were observed, indicating a progressive decline in motor performance of the HDtg.

Motor Performance Test in the Beam Walk Test

The motor performance in the beam walk test is illustrated in FIG. 15. The analysis of variance for repeated measurements reveal for the tests on a circular beam having a diameter of 16 mm a significant effect of the factor “genotype” in the time required to traverse the beam with F(2, 31): 8.1, p=0.0015 and the frequency of falling off with F(2, 31): 5.4, p=0.0096. Posthoc analysis by means of the PLSD test revealed that the statistical overall effect in this motor performance test was due to significant (p<0.0001) decline in the homozygous HDtg.

Discussion of the Behavioral Phenotyping (Example 3)

In the present invention we describe the first transgenic rat model for the human HD, which reflects very closely the frequent adult form of the human disease. HDtg rats manifest a slowly progressive phenotype with emotional alterations, cognitive disorders and reduced motor performance.

In the phenotype, HDtg rats are indistinguishable from their littermates, except for occasional dyskinetic movements of the head. The monitoring of the body weight provides evidence for the differential effect of the transgene on the growth rate and a slow progression of the disease, associated by an increasing loss of body weight. In addition, the Kaplan-Meier analysis provides evidence for the effect of the transgene on the survival rate of the HDtg rats in form of an increasing lethality as of the age of 18 months. These findings are in accordance with the human HD, since also the human disease entails cachexia and increased lethality.

One of the first behavioral abnormalities is a dramatically reduced anxiety of the HDtg rats in the elevated plus maze test of anxiety. This behavior test provides evidence for the differential effect of the transgene on the emotional parameter “anxiety” in HDtg rats, therefore comparable with findings in R6/2 mice (File et al., 1998) and the early emotional symptoms in HD patients. The finding in the plus maze is supported by similar effects in the social interaction test of anxiety. This test provides evidence for the effect of the transgene on the emotional parameter “anxiety” in a further behavior test which is unlike the elevated plus maze test of anxiety suited for repeated studies as progression tests for the emotional parameters. Interestingly, this reduced anxiety in the HDtg rats is not associated with increased exploration, as can be seen from the holeboard test of exploration. The holeboard test for exploration behavior provides evidence for the differential effect of the transgene in HDtg rats on the emotional/cognitive parameter “exploration”. Thus, the results are generally comparable with the first emotional symptoms in HD patients and could be defined as “pathological “fearlessness”.

Cognitive decline in the HDtg rats was first observed at the age of 10 months during the test for spatial learning in the 8-arm maze. The results provide evidence for a “working memory amnesia” which inhibits the formation of a “reference memory” (working memory deficiency), which also inhibits the generation of a reference memory (long term memory). This behavioral assay gives evidence for the effect of the transgene in HDtg rats on the cognitive parameter “spatial learning”, and is therefore comparable with findings in R6/2 mice and the cognitive symptoms in HD patients. Interestingly, associative learning tests with two-way-active-avoidance-transfer-compartments, which are performed under high levels of stress, revealed that the transgenic rats apparently have less difficulties in managing a simple associative learning task when subjected to stress. Such observations are frequently made for functional impairment of the hippocampus (Hölscher and Schmidt, 1998). The results of these behavioral assays could be pioneering for a deeper understanding of the pathology of the HD, since they indicate an important role of stress regulative systems and give evidence for a connection between the basal ganglia and the hippocampus. Cognitive decline is a common symptom in HD patients (Mohr et al., 1991). Early in the course of the disease, the patients show restrained spatial learning ability (Lawrence et al., 1996; 2000). Likewise, working memory dysfunction (Murphy et al., 2000) as well as other cognitive deficiencies (Lione et al., 1999) are also found in R6/2 mice.

Motor dysfunctions which are actually the reason for the naming of the human chorea Huntington occur both in the patient and in the present invention in full extent only at a later time. At the age of five months, the animals do not present any symptoms in the accelerod test, and only in the beam walk test, the homozygous animals present abnormalities, although there are already apparent emotional differences. The results of the accelerod test give evidence for a progressive decline of the motor performance of the HDtg rats. These progressive decline of the parameter “balance and motor coordination” can be found in a well validated behavioral assay which is also suited for repeated studies as progression control. The motor dysfunctions in the HDtg rats reach their full extent at a later time compared to the first occurrence of the emotional and/or cognitive alterations, which is a further similarity to the human HD. The differential motor dysfunctions at the age of five months among the heterozygous and the homozygous HDtg rats in the beam walk test give evidence that the onset and possibly also the specificity of the motor dysfunctions depend on the gene dose, since only the homozygous animals manifest a decline at that age.

The object of the present invention was to develop a transgenic animal model for the human HD, which reflects the late and slowly progressive course of the most frequent human form of the HD, and which allows for in vivo progression controls by means of neuroimaging in order to provide a basis for the evaluation of future therapeutic approaches. For the first time, such development was successful, using the endogenous rat HD promoter and a human cDNA with 51 CAG repeat units. It seems to be possible now to follow up disease progression and successful results of new therapies, e.g. by means of MRT and PET. Thus, the present invention has the potential for an important tool for the clarification of the pathomechanism and for testing future therapeutic approaches using long-term treatments, microsurgery, stem cell transplantation, or antisense treatments.

### REFERENCES

Ackermann R F, Lear J L (1989) Glycolysis-induced discordance between glucose metabolic rates measured with radiolabelled fluorodeoxyglucose and glucose. J Cereb Blood Flow Metab 9:774-785.
Almqvist E W, Bloch M, Brinkman R et al. (1999) A Worldwide Assessment of the Frequency of Suicide, Suicide Attempts, or Psychiatric Hospitalization after Predictive Testing for Huntington Disease. Am J Hum Genet 64:1293-1304
Auburger G, Ratzlaff T, Lunkes A et al. (1996) A gene for autosomal dominant paroxysmal choreoathetosis/spasticity (CSE) maps to the vicinity of a potassium channel gene cluster on chromosome 1p, probably within 2 cM between D1S443 and D1S197. Genomics 31:90-94
Benitez J, Robledo M, Ramos C et al. (1995) Somatic stability in chorionic villi samples and other Huntington fetal tissues. Hum Genet 96(2):229-232
Borlongan C V, Koutouzis T K, Freeman T B et al. (1995) Behavioral pathology induced by repeated systemic injections of 3-nitropropionic acid mimics the motoric symptoms of Huntington's disease. Brain Res 697:254-257.
Breivik T, Stephan M, Brabant G E et al. (2002) Postnatal LPS-induced illness predisposes to periodontal disease in adulthood. Brain Behav Immun http://www.idealibrary.com/servlet/doi/ 10.1006/brbi.2001.0642:
Brouillet E, Hantraye P, Ferrante R J et al. (1995) Chronic mitochondrial energy impairment produces selective striatal degeneration and abnormal choreiform movements in primates. Proc Natl Acad Sci USA 92:7105-7109.
Brouillet E (2000) Animal models of Huntington's disease: from basic research on neuronal death to assessment of new therapeutic strategies. Funct Neurol 15:239-251.
Brouillet E, Conde F, Beal M F et al. (1999) Replicating Huntington's disease phenotype in experimental animals. Prog Neurobiol 59:427-468.
Bruyn R P, Stoof J C (1990) The quinolinic acid hypothesis in Huntington's chorea. J Neurol Sci 95:29-38.
Burke J R, Enghild J J, Martin M E et al. (1996) Huntingtin and DRPLA proteins interact with the enzyme GAPDH. Nat Med 2:347-350
Carter R J, Lione L A, Humby T et al. (1999) Characterization of Progressive Motor Deficits in Mice Transgenic for the Human Huntington's Disease Mutation. J Neurosci 19(8):3248-3257
Chen M, Ona V O, Li M et al. (2000) Minocycline inhibits caspase-1 and caspase-3 expression and delays mortality in a transgenic mouse model of Huntigton disease. Nat Med 6, 797-801
Crawley J N (1999) Behavioral phenotyping of transgenic and knockout mice: experimental design and evaluation of general health, sensory functions, motor abilities, and specific behavioral tests. Brain Res 835:18-26.
Davies S W, Turmaine M, Cozens B A et al. (1997) Formation of Neuronal Intranuclear Inclusions Underlies the Neurological Dysfunction in Mice Transgenic for the HD Mutation. Cell 90:537-548
Dellen A van, Blakemore C, Deacon R, York D, Hannan A J (2000) Delaying the onset of Huntington's in mice. Nature 404:721-722
Demirkiran M, Jankovic J (1995) Paroxysmal dyskinesias: clinical features and classification. Ann Neurol 38:571-579
DiFiglia M, Sapp E, Chase K O et al. (1997) Aggregation of Huntingtin in Neuronal Intranuclear Inclusions and Dystrophic Neurites in Brain. Science 277:1990-1993
Dunnett S B, Carter R J, Watts C et al. (1998) Striatal transplantation in a transgenic mouse model of Huntington's disease. Exp Neurol 154:31-40.
File S E, Mahal A, Mangiarini L et al. (1998) Striking changes in anxiety in Huntington's disease transgenic mice. Brain Res 805:234-240.
Fink J, Rainier S, Wilkowski J et al. (1996) Paroxysmal dystonic choreoathetosis: tight linkage to chromosome 2q. Am J Hum Genet 59:140-145
Goldberg Y P, Kremer B, Andrew S E, et al. (1993) Molecular analysis of new mutations for Huntington's disease: intermediate alleles and sex of origin effects. Nat Genet 5:174-179
Goldberg Y P, Nicholson D W, Rasper D M, et al. (1996) Cleavage of huntingtin by apopain, a proapoptotic cysteine protease, is modulated by the polyglutamine tract. Nat Genet 13:442
Goodenough D J, Fariello R G, Annis B L, Chun R W M (1978) Familial and acquired paroxysmal dyskinesias: a proposed classification with delineation of clinical features. Arch Neurol 35:827-831
Guidelines for the molecular genetics predictive test in Huntington's disease. (1994) Neurology 44:1533-1536
Gutekunst C A, Li SH, Yi H et al. (1999) Nuclear and neuropil aggregates in Huntington's disease: relationship to neuropathology. J Neurosci 19:2522-2534.
Hardie R J, Pullon H W, Harding A E et al. (1982)“New” clinical features of Mc Leod syndrome. Transfusion 22:404
Harper P S (1992) The epidemiology of Hutington's disease. Hum Genet 89:365-376
Harper P S. Huntington's Disease, (W.B. Saunders, London, 1996).
Hayden M R, Martin W R, Stoessl A J et al. (1986) Positron emission tomography in the early diagnosis of Huntington's disease. Neurology 36:888-894.
Hennig J, Nauerth A, Friedburg H (1986) RARE imaging: a fast imaging method for clinical MR. Magn Reson Med 3:823-833.
Hodgson J G, Agopyan N, Gutekunst C A et al. (1999) A YAC mouse model for Huntington's disease with full-length mutant huntingtin, cytoplasmic toxicity, and selective striatal neurodegeneration. Neuron 23:181-192.
Hockly E, Cordery P M, Woodman B, et al. (2002) Environmental enrichment slows disease progression in R6/2 Huntington's disease mice. Ann Neurol 51:235-242
Holbert S, Denghien I, Kiechle T et al. (2001) The Gln-Ala repeat transcriptional activator CA150 interacts with huntingtin: Neuropathologic and genetic evidence for a role in Huntington's disease pathogenesis. PNAS 98:1811-1816
Holscher C, Schmidt W J (1994) Quinolinic acid lesion of the rat entorhinal cortex pars medialis produces selective amnesia in allocentric working memory (WM), but not in egocentric WM. Behav Brain Res 63:187-194.
Holzmann C, Maueler W, Petersohn D et al. (1998) Isolation and characterization of the rat huntingtin promoter. Biochem J 336:227-234.
Jacobs R E, Cherry S R (2001) Complementary emerging techniques: high-resolution PET and MRI. Curr Opin Neurobiol 11:621-629.
Kambouris M, Bohlega S, Al-Tahan A, Meyer B F (2000) Localization of the Gene for a Novel Autosomal Recessive Neurodegenerative Huntington-Like Disorder to 4p15.3. Am J Hum Genet 66:445-452
Kompoliti K (1999) Estrogen and movement disorders. Clin Neuropharmacol 22 (6): 318-326.
Kremer B, Squitieri F, Telenius H, Andrew S E, Theilmann J, Spence N, Goldberg Y P, Hayden M R (1993) Molecular analysis of late onset Huntington's disease. J Med Genet 30:991-995
Kremer B, Goldberg P, Andrew S E et al. (1994) A worldwide study of the Huntington's disease mutation. New Engl J Med 330:1401-1406
Kuge Y, Minematsu K, Hasegawa Y et al. (1997) Positron emission tomography for quantitative determination of glucose metabolism in normal and ischemic brains in rats: an insoluble problem by the Harderian glands. J Cereb Blood Flow Metab 17:116-120.
Kuhl D E, Phelps M E, Markham C H et al. (1982) Cerebral metabolism and atrophy in Huntington's disease determined by 18FDG and computed tomographic scan. Ann Neurol 12:425-434.
Kuwert T, Lange H W et al. (1990) Cortical and subcortical glucose consumption measured by PET in patients with Huntington's disease. Brain 113: 1405-1423.
Lawrence A D, Sahakian B J, Hodges J R et al. (1996) Executive and mnemonic functions in early Huntington's disease. Brain 119:1633-1645.
Lawrence A D, Watkins L H, Sahakian B J et al. (2000) Visual object and visuospatial cognition in Huntington's disease: implications for information processing in corticostriatal circuits. Brain 123:1349-1364.
Li H, Li SH, Cheng A L et al. (1999) Ultrastructural localization and progressive formation of neuropil aggregates in Huntington's disease transgenic mice. Hum Mol Genet 8:1227-1236.
Li H, Li SH, Johnston H et al. (2000) Amino-terminal fragments of mutant huntingtin show selective accumulation in striatal neurons and synaptic toxicity. Nat Genet 25:385-389.
Lione L A, Carter R J, Hunt M J et al (1999) Selective Discrimination Learning Impairments in Mice Expressing the Human Huntington's Disease Mutation. J Neurosci 19(23): 10428-10337 Lüiesse H-G, Schiefer J, Spruenken A et al. (2001) Evaluation of R6/2 HD transgenic mice for therapeutic studies in Huntington's disease: behavioral testing and impact of diabetes mellitus. Beh. Brain Res. in press:
Lyle O E, Gottesman II (1977) Premorbid psychometric indicators of the gene for Huntington's disease. J Consult Clin Psychol 45: 1011-1022.
Malandrini A, Fabrizi G M, Truschi F et al. (1994) Atypical McLeod syndrome manifested as X-linked chorea-acanthocytosis, neuromyopathy and dilated cardiomyopathy: report of a family. J Neurol Sci 124:89-94
Mangiarini L, Sathasivam K, Seller M et al. (1996) Exon 1 of the HD Gene with an Expanded CAG Repeat Is Sufficient to Couse a Progressive Neurological Phenotype in Transgenic Mice. Cell 87:493-506
Margolis R L, O'Hearn E, Rosenblatt A et al. (2001) A disorder similar to Huntington's disease ist associated with a novel CAG repeat expansion. Ann Neurol in press
Martin J B, Gusella J F (1986) Huntington's disease: Pathogenesis and management. New Engl J Med 315:1267-1276
Mohr E, Brouwers P, Claus J J et al. (1991) Visuospatial cognition in Huntington's disease. Mov Disord 6:127-132.
Moore T H, Osteen T L, Chatziioannou T F et al. (2000) Quantitative assessment of longitudinal metabolic changes in vivo after traumatic brain injury in the adult rat using FDG-microPET. J Cereb Blood Flow Metab 20:1492-1501.
Mullins J J, Peters J, Ganten D (1990) Fulminant hypertension in transgenic rats harbouring the mouse Ren-2 gene. Nature 344:541-544.
Murphy K P, Carter R J, Lione L A et al. (2000) Abnormal synaptic plasticity and impaired spatial cognition in mice transgenic for exon 1 of the human Huntington's disease mutation. J Neurosci 20:5115-5123.
Ona V O, Li M, Vonsattel J P G et al. (1999) Inhibition of caspase-1 slows disease progression in a mouse model of Huntington's disease. Nature 399:263-267
Paulson H L, Fischbeck K H (1996) Tri-nucleotide repeats in neurogenetic disorders. Annu Rev Neurosci 19:79-107.
Peters M F, Ross C A (2001) Isolation of a 40 kDa Huntingtin-Associated Protein. J Biol Chem 276:3188-3194
Phelps M E (2000) PET: The merging of biology and imaging into molecular imaging. J. Nucl. Med. 41:661-681.
Pietrzyk U, Herholz K, Heiss W D (1990) Three-dimensional alignment of functional and morphological tomograms. J Comput Assist Tomogr 14:51-59.
Provencher S W (1993) Estimation of metabolite concentrations from localized in vivo proton NMR spectra. Magn Reson Med 30:672-679.
Quarrell O W, Youngman S, Sarfarazi M, Harper P S (1988) Absence of close linkage between benign hereditary chorea and the locus D4S10 (probe G8). J Med Genet 25:191-194
Rampoldi L, Dobson-Stone C, Rubio J P et al. (2001) A conserved sorting-associated protein is mutant in chorea-acanthocytosis. Nat Genet 28:119-120
Reddy P H, Williams M, Charles V et al. (1998) Behavioural abnormalities and selective neuronal loss in HD transgenic mice expressing mutated full-length HD cDNA. Nat Genet 20:198-202.
Rubinsztein D C, Leggo J, Coles R et al. (1996) Phenotypic characterization of individuals with 30-40 CAG repeats in the Huntington disease (HD) gene reveals HD cases with 36 repeats and apparently normal elderly individuals with 36-39 repeats. Am J Hum Genet 59:16-22
Rubinsztein D C, Leggo J, Chiano M et al. (1997) Genotypes at the GluR6 kainate receptor locus are associated with variation in the age of onset of Huntington disease. Proc Natl Acad Sci USA 94:3872-3876
Russo D, Redman C, Lee S (1998) Association of XK and Kell blood group proteins. J Biol Chem 273:13950-13956
Saudou F, Finkbeiner S, Devys D, Greenberg M E (1998) Huntingtin Acts in the Nucleus to induce Apoptosis but Death Does Not Correlate with the Formation of Intranuclear Inclusions. Cell 95:55-66
Scherzinger E, Lurz R, Turmaine M et al. (1997) Huntingtin-Encoded Polyglutamine Expansions Form Amyloid-like Protein Aggregates in vitro and in vivo. Cell 90:549-558
Schilling G, Becher M W, Sharp A H et al. (1999) Intranuclear inclusions and neuritic aggregates in transgenic mice expressing a mutant N-terminal fragment of huntingtin. Hum Mol Genet 8:397-407.
Schinke M, Baltatu O, Bohm M et al. (1999) Blood pressure reduction and diabetes insipidus in transgenic rats deficient in brain angiotensinogen. Proc Natl Acad Sci USA 96:3975-3980.
Schmidt T, Landwehrmeyer G B, Schmitt I et al. (1998) An isoform of ataxin-3 accumulates in the nucleus of neuronal cells in affected brain regions of SCA3 patients. Brain Pathol 8:669-679.
Schmitt I, Bachner D, Megow D et al. (1995) Expression of the Huntington disease gene in rodents: cloning the rat homologue and evidence for downregulation in non-neuronal tissues during development. Hum Mol Genet 4:1173-1182.
Schoenfield M, Myers R H (1984) Increased rate of suicide among patients with Huntington's disease. J Neurol Neurosurg Psychiat 47: 1283.
Schoppe K J (1974) Das MLS-Gerät: Ein neuer Testapparat zur Messung feinmotorischer Leistungen. Diagnostica 20:43-46.
Shelbourne P F, Killeen N, Hevner R F et al. (1999) A Huntington's disease CAG expansion at the murine Hdh locus is unstable and associated with behavioural abnormalities in mice. Hum Mol Genet 8:763-774.
Shinotoh H, Calne D B, Snow B, Hayward M, Kremer B, Theilmann J, Hayden M R (1994) Normal CAG repeat length in the Huntington's disease gene in senile chorea. Neurology 44:2183-2184
Sittler A, Lurz R, Lueder G et al. (2001) Geldanamycin activates a heat shock response and inhibits huntingtin aggregation in a cell culture model of Huntington's disease. Hum Mol Genet 10:1307-1315
Sokoloff L, Reivich M, Kennedy C et al. (1977) The [14C]deoxyglucose method for the measurement of local cerebral glucose utilization: theory, procedure, and normal values in the conscious and anesthetized albino rat. J Neurochem 28:897-916.
Stone T W (2001) Kynurenines in the CNS: from endogenous obscurity to therapeutic importance. Prog Neurobiol 64:185-218.
Swerdlow N R, Geyer M A (1998) Using an animal model of deficient sensorimotor gating to study the pathophysiology and new treatments of schizophrenia. Schizophr Bull 24:285-301.
Szepetowski P, Rochette J, Berquin P et al. (1997) Familial Infantile Convulsions and Paroxysmal Choreoathetosis: A New Neurological Syndrome Linked to the Pericentromeric Region of Human Chromosome 16. Am J Hum Genet 61:889-898
Telenius H, Kremer H P H, Theilmann J et al. (1993) Molecular analysis of juvenile Huntington disease: the major influence on (CAG)n repeat length is the sex of the affected parent. Hum Mol Genet 2:1535-1540
Telenius H, Kremer B, Goldberg Y P et al. (1994) Somatic and gonadal mosaicism of the Huntington disease gene CAG repeat in brain and sperm. Nat Genet 6(4):409-414
Telenius H, Almquist E, Kremer B et al. (1995) Somatic mosaicism in sperm is associated with intergenerational (CAG)n changes in Huntington disease. Hum Mol Genet 4:189-195
The Huntington's disease collaborative research group (1993) A novel gene containing a tri-nucleotide repeat that is expanded and unstable on Huntington's disease chromosomes. Cell 72:971-983
Tomita H, Nagamitsu S, Wakui K et al (1999) Paroxysmal kinesigenic choreoathetosis locus maps to chromosome 16p11.2-q12.1. Am J Hum Genet 65:1688-1697
Tzagoumissakis M, Fesdjian C, Shashidharan P, Plaitakis A (1995) Stability of the Huntington disease (CAG)ₙ repeat in a late-onset form occurring on the Island of Crete. Hum Mol Genet 4:2239-2243
Ueno S, Maruki Y, Nakamura M et al. (2001) The gene encoding a newly discovered protein, chorein, is mutated in chorea-acanthocytosis. Nat Genet 28:121-122
Vaarmann A, Kask A, Mäeorg U (2002) A novel and sensitive HPLC method for determination of catecholamine and kynurenine derivatives of tryptophan using couulometric array detector. J. Chromatography 769:145-153.
Vries B B A de, Arts W F M, Breedveld G J et al. (2000) Benign Hereditary Chorea of Early Onset Maps to Chromosome 14q. Am J Hum Genet 66:136-142
Warner J P, Barron L H, Brock D J H (1993) A new polymerase chain reaction (PCR) assay for the tri-nucleotide repeat that is unstable and expanded on Huntington's disease chromosomes. Mol Cell Probes 7:235-239
Warner T T, Lennox G G (1994) Autosomal-dominant dentatorubropallidoluysian atrophy in the United Kingdom. Mov Disord 9: 289-296.
Warrick J M, Chan H Y E, Gray-Board G L et al. (1999) Suppression of polyglutamine-mediated neurodegeneration in Drosophila by the molecular chaperone HSP70. Nat Genet 23:425-428
Weindl A, Conrad B (1996) Chorea und choreatische Bewegungsstorungen, in: Conrad B, Ceballos-Baumann A O: Bewegungsstörungen in der Neurologie. Thieme 1996: 155-180.
Weber S, Bauer A, Herzog F et al. (2000) Recent Results of the TierPET Scanner. IEEE Trans. Nucl. Sci. 47:1665-1669.
Wheeler V C, White J K, Gutekunst C A et al. (2000) Long glutamine tracts cause nuclear localization of a novel form of huntingtin in medium spiny striatal neurons in HdhQ92 and HdhQ111 knock-in mice. Hum Mol Genet 9:503-513.
Widner B, Werner E R, Schennach H et al. (1999) An HPLC method to determine tryptophan and kynurenine in serum simultaneously. Adv Exp Med Biol 467:827-832.
Yamamoto A, Lucas J J, Hen R (2000) Reversal of neuropathology and motor dysfunction in a conditional model of Huntington's disease. Cell 101:57-66.
Young AB, Penney J B, Starosta-Rubinstein S et al. (1986) PET scan investigations of Huntington's disease: cerebral metabolic correlates of neurological features and functional decline. Ann Neurol 20:296-303Xiang F, Almqvist E W, Huq M et al. (1998) A Huntington Disease-Like Neurodegenerative Disorder Maps to Chromosome 20p. Am J Hum Genet 63:1431-1438
Zuccato C, Ciammola A, Rigamonti D et al. (2001) Loss of Huntingtin-Mediated BDNF Gene Transcription in Huntington's Disease. Science 14: in press Zühlke C, Riess O, Schröder K, et al. (1993) Expansion of the (CAG)ₙ repeat causing Huntington's disease in 352 patients of German origin. Hum Mol Genet 2:1467-1469

1-15. (canceled)

16. A nucleic acid construct comprising:
a carboxy-terminally truncated sequence of the rat huntingtin gene (RHT 10),
at least 36 tri-nucleotide repeats, and
further upstream at least an effective portion of a huntingtin gene specific promoter.

17. The nucleic acid construct according to claim 16, wherein the tri-nucleotide repeats are present within a human huntingtin gene portion integrated into the construct, wherein the rat huntingtin gene was truncated N-terminally for the CAG repeat region.

18. The nucleic acid construct according to claim 17, wherein the rat huntingtin gene was truncated N-terminally for 154 base pairs.

19. The nucleic acid construct according to claim 17, wherein the aberrant human huntingtin gene portion that was integrated into the nucleic acid construct having CAG tri-nucleotide repeats was obtained by a PCR reproduction with the primers Hu4 (ATGGC GACCCTGGAAAAGCTGATGAA) and Hu3-510 (GGGCGCCTGAGGCTGAGGCAGC) from the DNA of a Chorea Huntington patient.

20. The nucleic acid construct according to claim 16, wherein the construct comprises a poly-adenylation sequence downstream from the rat Huntington gene.

21. The nucleic acid construct according to claim 16, wherein the construct comprises at least 36 CAG tri-nucleotide repeats.

22. The nucleic acid construct according claim 16, wherein the huntingtin gene specific promoter is a promoter expressing in the brain.

23. The nucleic acid construct according claim 22, wherein the gene specific promoter is chosen from a native rat huntingtin promoter or a functional portion thereof.

24. The nucleic acid construct according claim 16, wherein the functional rat huntingtin gene fragment is a proportion of the sequence according to GenBank accession No U18650.

25. A Vector comprising the nucleic acid construct according to claim 16.

26. A mammalian cell, excluding an embryonic human stem cell, transfected with the nucleic acid construct according to the vector of claim 25.

27. A transgenic rat comprising in the genome of its germ line cells and somatic cells an aberrant sequence of the rat huntingtin gene expanded by CAG repeat units, which have been introduced into this animal or into one of its predecessors.

28. The transgenic rat according to claim 27, wherein the gene sequence comprises at least 36 tri-nucleotide repeats, wherein the number of repeats is 40.

29. The transgenic rat according to claim 27, wherein the gene sequence comprises at least 36 tri-nucleotide repeats, wherein the number of repeats is 50 to 200.

30. The transgenic rat according to claim 27, wherein a nucleic acid construct has been introduced into the rat or into one of its predecessors,
wherein the nucleic acid construct comprises:
a carboxy-terminally truncated sequence of the rat huntingtin gene (RHT 10),
at least 36 tri-nucleotide repeats, and
further upstream at least an effective portion of a huntingtin gene specific promoter.

31. An use of the transgenic rat according to claim 27 as a model animal for performing studies on the pathomechanism and progress of the disease Chorea Huntington and other neurodegenerative diseases of the central nervous system, for the development of therapeutic and/or prophylactic agents against these diseases, for the examination of therapy concepts, for performing microsurgical surgery, stem cell transplantations or gene therapeutic treatments or anti-sense treatments and for the progress control using imaging processes, especially PET and MRI.
